(19) United States
(12) Patent Application Publication
(10) Pub. No.: US 2024/0247315 A1
(43) Pub. Date: Jul. 25, 2024

(54) DIAGNOSING INFLAMMATORY BOWEL DISEASES

(71) Applicants: YEDA RESEARCH AND DEVELOPMENT CO. LTD., Rehovot (IL); SHEBA IMPACT LTD., Ramat-Gan (IL)

(72) Inventors: Shaul Shalev Itzkovitz, Rehovot (IL); Keren Bahar Halpern, Rehovot (IL); Adi Egozi, Rehovot (IL); Shomron Silan Ben-Horin, Ramat-Efal (IL); Bella Ungar, Ramat Gan (IL); Shani Ben-Moshe, Rehovot (IL)

(21) Appl. No.: 18/577,231

(22) PCT Filed: Jul. 21, 2022
(86) PCT No.: PCT/IL2022/050793

§ 371 Date: Jan. 5, 2024

(30) Foreign Application Priority Data
Jul. 21, 2021 (IL) 285031

(51) Int. Cl. C12Q 1/6883

(52) U.S. Cl. CPC C12Q 1/6883; C12Q 2600/136; C12Q 2600/158

(57) ABSTRACT

A method of diagnosing an inflammatory bowel disease (IBD) of a subject is disclosed. The method comprises analyzing the RNA expression level of particular human gene in a fecal RNA sample of the subject, wherein when the expression level is above a predetermined amount it is indicative of the inflammatory bowel disease.

### RELATED APPLICATION

This application claims the benefit of priority of Israel Patent Application No. 285031 filed 21 Jul., 2021, the contents of which are incorporated herein by reference in their entirety.

### SEQUENCE LISTING STATEMENT

The file entitled 92757.xml, created on 21 Jul. 2022, comprising 53,248 bytes, submitted concurrently with the filing of this application is incorporated herein by reference.

### FIELD AND BACKGROUND OF THE INVENTION

The present invention, in some embodiments thereof, relates to methods of diagnosing gastric diseases and more particularly inflammatory bowel diseases.

Biologic therapies have revolutionized therapy for moderate to severe IBD. While 50-60% of patients significantly improve with biologics and experience less hospitalizations and surgeries, many patients are either primary non-responders or experience loss of response over time. Non-invasive markers that may provide information on histological inflammation, and therefore predict patient prognosis or response to therapies, are critically needed.

Several studies performed RNA sequencing of colonic biopsies obtained during lower endoscopies, with the aim of staging the disease and predicting therapeutic outcomes. Furthermore, certain mucosal micro-RNA and long noncoding RNA have been associated with IBD natural history. Recent studies used single cell RNA sequencing (scRNAseq) and single cell mass-cytometry of IBD biopsy samples to reveal distinct populations and genes that are altered in specific disease states. In addition to transcriptomics, unique DNA methylation patterns have been identified in biopsies of IBD patients compared to controls. Data from RNA bulk sequencing of intestinal biopsies has also been integrated with genome-wide-associations to identify genes most associated with regulatory pathways in IBD. Nevertheless, an outstanding challenge of the analysis of biopsies is that they provide localized information and may miss out on inflammatory processes, especially in cases where endoscopic inflammation is not apparent.

A complementary method to assess intestinal inflammation is the use of fecal samples. A recent study demonstrated that patients with active Crohn's disease had a distinct microRNA profile measured in their stool. Fecal proteomics can also inform on intestinal inflammation status. Indeed, calprotectin, a leukocyte protein, is a widely applied biomarker of intestinal inflammation. Nevertheless, the calprotectin assay is limited in sensitivity and specificity and only few additional proteins have been shown to be both resistant to proteolysis and associated with inflammation. An advantage of fecal samples is that they may provide broad sampling of processes that occur throughout the gastrointestinal tract. Recent works demonstrated that fecal host transcriptomes may carry prognostic information related to colorectal cancer, however the utility of this approach to the staging and prognosis of IBDs has not been explored.

Background art includes Cui et al., Digestive Diseases and Sciences (2021) 66: 1488-1498; and US Patent Application No. 20200308644.

### SUMMARY OF THE INVENTION

According to an aspect of the present invention there is provided a method of diagnosing an inflammatory bowel disease (IBD) of a subject comprising analyzing the RNA expression level of at least one human gene in a fecal RNA sample of the subject, wherein the gene is selected from the group consisting of CSF3R, CASP4, NFKB1A, CFLAR, FAM49B, RNF145, FOSL2, PEL1, PTPRE, GK. MX2, NAGK, MCTP2, SLCO3A1, STAT1, RASSF3, MARCKS, SAT1, VPS37B, RNF149, HLA-E, PLAUR, MSN, HIF1A, NBPF14, CXCR1, CSF2RA, CLEC2B, GBP5, IL1B, FZD3, MMP25 and OSM wherein when the expression level is above a predetermined amount it is indicative of the inflammatory bowel disease.

According to an aspect of the present invention there is provided a method of diagnosing a disease of the gastrointestinal tract of a subject comprising analyzing the expression level of at least one gene in a fecal wash of the subject, wherein the expression level is indicative of the disease of the gastrointestinal tract.

According to an aspect of the present invention there is provided a method of diagnosing an inflammatory bowel disease (IBD) of a subject comprising analyzing the RNA expression level of at least one human gene in a fecal RNA sample of the subject, wherein when the expression level of a human gene set forth in Table 1, 5 or 6 is statistically significantly altered over the level of the gene in a fecal RNA sample of a control subject, it is indicative of the inflammatory bowel disease.

According to embodiments of the invention, the expression level of the at least one gene correlates with the degree of histological inflammation.

According to an aspect of the present invention there is provided a method of treating an inflammatory bowel disease of a subject in need thereof comprising:

(a) confirming that the subject has the inflammatory bowel disease according to the method described herein; and

(b) administering to the subject a therapeutically effective amount of an agent useful for treating the disease.

According to an aspect of the present invention there is provided a method of treating a disease of the gastrointestinal tract of a subject in need thereof comprising:

(a) confirming that the subject has the inflammatory bowel disease according to the method described herein; and

(b) administering to the subject a therapeutically effective amount of an agent useful for treating the disease.

According to an aspect of the present invention there is provided a method of selecting an agent for the treatment of an inflammatory bowel disease (IBD) comprising:

(a) contacting the agent with an RNA sample derived from feces of a subject having the IBD; and

(b) analyzing the amount of at least one RNA set forth in Table 1, wherein a decrease in the amount of the at least one RNA in the presence of the agent as compared to the amount of the at least one RNA in the absence of the agent is indicative of an agent which is suitable for the treatment of the inflammatory bowel disease.

According to embodiments of the invention, the method further comprises depleting the fecal RNA sample of microbial RNA prior to the analyzing.

According to embodiments of the invention, the fecal wash is of the sigmoid colon of the subject.

According to embodiments of the invention, the fecal wash is of the rectum of the subject.

According to embodiments of the invention, the analyzing the expression level comprises performing whole cell transcriptome analysis.

According to embodiments of the invention, the analyzing the expression level comprises performing RT-PCR.

According to embodiments of the invention, the analyzing is effected at the RNA level.

According to embodiments of the invention, the analyzing is effected at the protein level.

According to embodiments of the invention, the fecal sample comprises a fecal wash, the at least one gene is selected from the group consisting of CSF3R, CFLAR, FAM49B, MX2, STAT1, CASP4, NFKB1A, RNF145, FOSL2, PEL1, PTPRE and GK.

According to embodiments of the invention, the at least one gene is selected from the group consisting of CSF3R, CASP4, NFKB1A, RNF145, FOSL2, PEL1, PTPRE, MX2, NAGK, MCTP2. SLCO3A1, STAT1, RASSF3 and GK.

According to embodiments of the invention, the at least one gene is selected from the group consisting of MX2, CSF3R, NAGK, MCTP2, SLCO3A1, CASP4, NFKBIA, STAT1, RNF145 and RASSF3.

According to embodiments of the invention, the fecal sample comprises a solid fecal sample, the at least one gene is selected from the group consisting of MARCKS, SAT1, NFKBIA, VPS37B. RNF149, HLA-E, PLAUR, MSN, HIF1A, NBPF14, CXCR1, CSF2RA, CLEC2B, GBP5, IL1B, FZD3, MMP25 and OSM.

According to embodiments of the invention, the disease is an inflammatory bowel disease (IBD).

According to embodiments of the invention, the IBD comprises ulcerative colitis or Crohn's colitis.

According to embodiments of the invention, the disease is a colon cancer.

According to embodiments of the invention, the disease is irritable bowel syndrome.

According to embodiments of the invention, the diagnosing the IBD comprises determining the severity of the IBD.

According to embodiments of the invention, the RNA sample is a solid fecal sample, the at least one gene is selected from the group consisting of MARCKS, SAT1, NFKBIA, VPS37B, RNF149, HLA-E, PLAUR, MSN, HIF1A, NBPF14, CXCR1, CSF2RA, CLEC2B, GBP5, IL1B, FZD3, MMP25 and OSM.

According to embodiments of the invention, the RNA sample is a fecal wash of the subject, the at least one gene is selected from the group consisting of CSF3R, CFLAR, FAM49B, MX2, STAT1, CASP4, NFKB1A, RNF145, FOSL2, PEL1, PTPRE and GK.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1—Illustration of experimental layout.

FIGS. 2A-E—Fecal wash gene expression patterns are more indicative of histological inflammation compared to those of biopsies. A—Principal Component Analysis (PCA) plot showing biopsies (blue circles) and fecal washes (brown circles). Red outer circles denote samples that correspond to patients with histological inflammation determined based on pathology examination of the colonic biopsies. B—Hierarchical clustering of fecal wash samples (brown branches) and colonic biopsies (blue branches). Samples corresponding to patients with active histological inflammation are marked in red. Naming nomenclature: sample name-condition-endoscopic inflammation (0/1)—histological inflammation (0/1). C. D—PCA plots of biopsies (C), and fecal wash samples (D). Red outer circles denote IBD patients with corresponding histological inflammation. E—Transcriptomic signatures of fecal washes for patients with histological inflammation are more correlated among themselves than those of biopsy transcriptomic signatures. Violin plots demonstrating that the correlation distances between pairs of samples that both have histological inflammation (red dots) are significantly smaller than the distances between mixed samples with and without histological inflammation when examining fecal washes (brown dots, bottom) but not when examining biopsies (blue dots, top). White circles are medians, black boxes denote the 25-75 percentiles.

FIGS. 3A-C—Differentially expressed genes between inflamed and non-inflamed fecal washes. A—Volcano plot, each dot is a gene, x-axis is the log2-ratio of expression between samples with and without histological inflammation, y axis is −log10 (p value), where p value is computed using Wilcoxon rank sum tests. Genes with corresponding q-values below 0.1 are marked in red (q-values computed using Benjamini-Hochberg FDR correction). Names of representative up-regulated genes are shown. B—Hierarchical clustering of fecal wash samples over 100 genes consisting of 50 genes with the maximal ratio of expression levels and 50 with the lowest ratio between histologically inflamed and non-inflamed washes. Samples corresponding to patients with active histological inflammation are marked with red branches. C—Gene Set Enrichment Analysis (GSEA) over the Hallmark and Kegg sets. Shown are all gene sets with q-value<0.3. Inflamed washes (red circles) were associated with immune cell pathways, while non-inflamed washes (blue circles) expressed more epithelial cell related pathways. Naming nomenclature: sample name-condition-endoscopic inflammation (0/1)—histological inflammation (0/1).

FIGS. 4A-B—Cell compositions of inflamed versus non inflamed fecal washes and biopsies, inferred by computational deconvolution. A—Hierarchical clustering of cell type representation in fecal wash samples and colonic biopsies. Fecal washes from patients with histological inflammation are marked in red. B—Inferred relative representation of genes associated with different cell types in histologically inflamed and non-inflamed colonic biopsies and fecal washes. Immune-related cell types, more abundant in the fecal washes of patients with histological inflammation, are marked with a red box. Naming nomenclature: sample name-condition-endoscopic inflammation (0/1)—histological inflammation (0/1). White circles are medians, gray boxes denote the 25-75 percentiles.

FIGS. 5A-E—Expression of individual genes in fecal washes has a higher statistical power in classifying histological inflammation compared to biopsy gene expression. A—ROC curve example for the gene NFKBIA using fecal washes (blue, AUC=0.97) and biopsies (red, AUC=0.67). B—AUC of 5% genes with the highest AUC for biopsies and washes. The AUC of the top classifier genes is significantly higher for fecal washes compared to biopsies (p=1.85*10⁻⁷²). C—Comparison of AUC for individual genes based on biopsies (X axis) and fecal washes (Y axis). NFKBIA (black dot) is shown as an example. Gray boxes mark the top AUC (>0.9) for both groups. Fecal washes contain 150 genes with AUC>0.9 whereas biopsies contain only 10 such genes. D. E—Expression levels for the eight genes with the highest AUC levels for washes (D) and biopsies (E). White circles are medians, gray boxes mark the 25-75 percentiles.

FIGS. 6A-B—Protein and mRNA levels in fecal washes are only weakly correlated. A—Fecal calprotectin levels as measured by a commercial ELISA assay are correlated with the Mass-Spectrometry proteomics levels of the same protein, S100A8, S100A9. Each blue dot denotes a fecal sample. B—Correlation between protein levels and fecal wash mRNA levels for the same samples. Each dot is the average expression over the four samples.

FIGS. 7A-F—Analysis of the Spearman correlation distances between pairs of washes (A,C,E)/biopsies (B,D.F) with endoscopic inflammation (A-B) or histological inflammation (C-F). C-D—Analysis stratified over patient ages between 40 and 60 only. E-F—Analysis stratified for patients not receiving biologics. White circles are medians, black boxes denote the 25-75 percentiles.

FIG. 8—Analysis of the Spearman correlation distance between each wash and its matching biopsy in comparison to the mean of the distances to other biopsies. In 23 out of 31 samples the distance to other biopsies was higher. (only samples with more than 10000 Unique Molecular Identifiers (UMIs) in both washes and biopsies were included, FIGS. 7A-F).

FIG. 9—Clusters of human colonic cell types based on a recent single cell RNAseq atlas. The average expression of each cluster was used as input signature for CIBERSORTx computational deconvolution.

FIGS. 10A-E illustrate that bacterial rRNA depleted stool transcriptomics are informative in assessing intestinal inflammation. A. Box plots of fractions of reads mapped to human exonic regions from seven samples, before depleting bacterial rRNA (left) and after depleting bacterial rRNA (right). Red lines denote group medians, blue boxes mark IQR. Gray horizontal lines mark the change within each sample. Ranksum paired test p-value=0.0156. B. Principal component analysis (PCA) on transcriptomes of 106 wash samples (above 10000 UMIs) and 7 stool samples (above 7500 UMIs). Included in the analysis are genes with maximal expression level of at least 0.005 across all samples. C. Clustergram of 106 wash samples and 7 stool samples. Red branches mark a cluster enriched with inflamed samples. Inflamed washes are colored pink, non-inflamed washes are colored blue, inflamed stools are colored brown and non-inflamed stools are colored turquoise. D. differential gene expression between stool samples of inflamed IBD patients (n=3 samples) and of non-inflamed individual (n=7 samples). All samples included in the analysis had more than 5000 UMIs. Grey dots demarcate all genes used for the analysis. Dots encircled in red and blue are genes upregulated and downregulated in IBD inflamed fecal washes, respectively (|fold change|>1.5, FDR<0.01). Venn diagrams at the top of the plot demonstrate the overlap between genes downregulated (top left) or upregulated (top right) in both washes and stool samples of inflamed IBD patients, together with the number of non-overlapping genes in each sampling method. P-values were calculated using hypergeometric test. E. Violin plots of selected genes with different expression in inflamed (n=3) and non-inflamed (n=7) stool samples. White dots mark the group median, purple lines are plotted between the means of the groups.

### DESCRIPTION OF SPECIFIC EMBODIMENTS OF THE INVENTION

The present invention, in some embodiments thereof, relates to methods of diagnosing gastric diseases and more particularly inflammatory bowel diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Colonoscopy is the gold standard for evaluation of inflammation in inflammatory bowel disease (IBD), yet entails cumbersome preparations and risks of injury. Existing non-invasive prognostic tools are limited in their diagnostic power. Moreover, transcriptomics of colonic biopsies have been inconclusive in their association with clinical features.

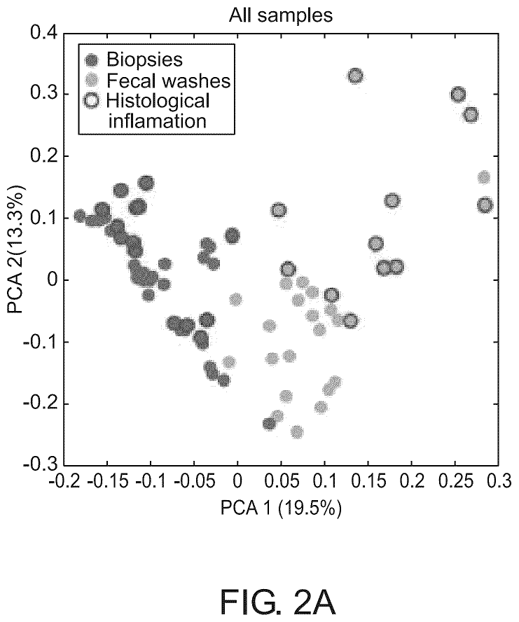

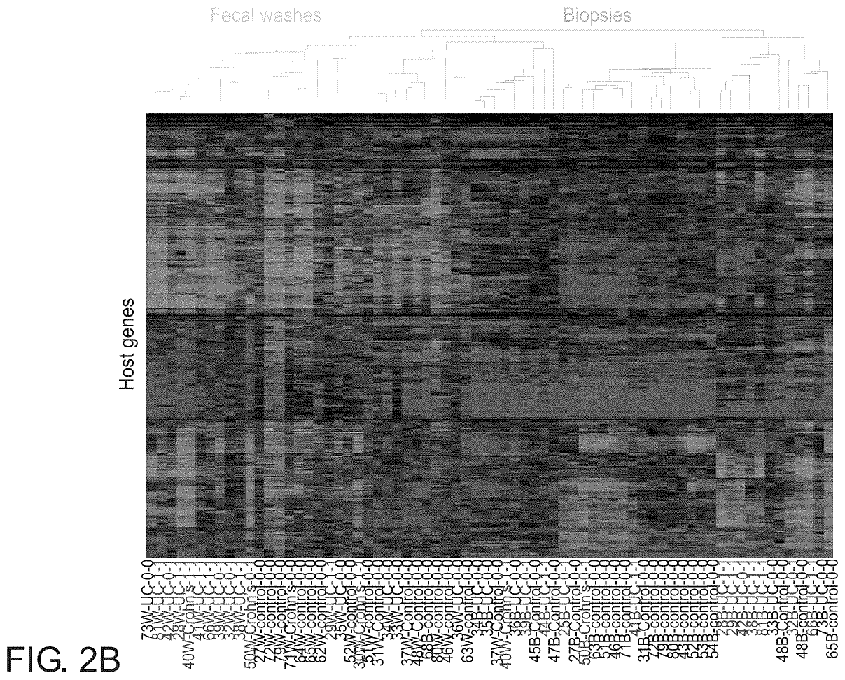

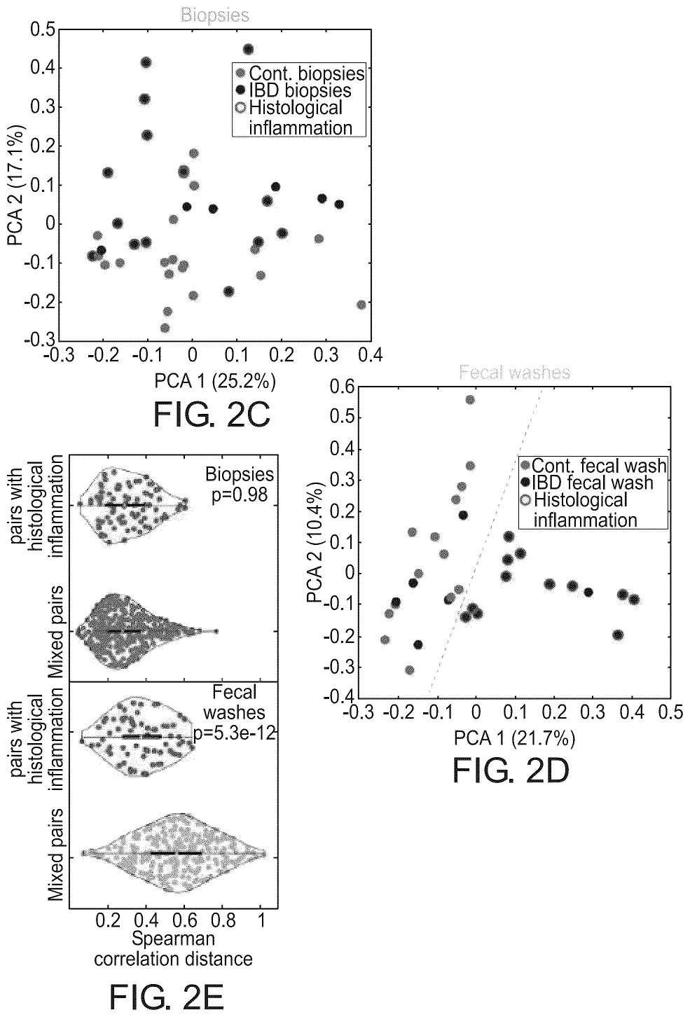

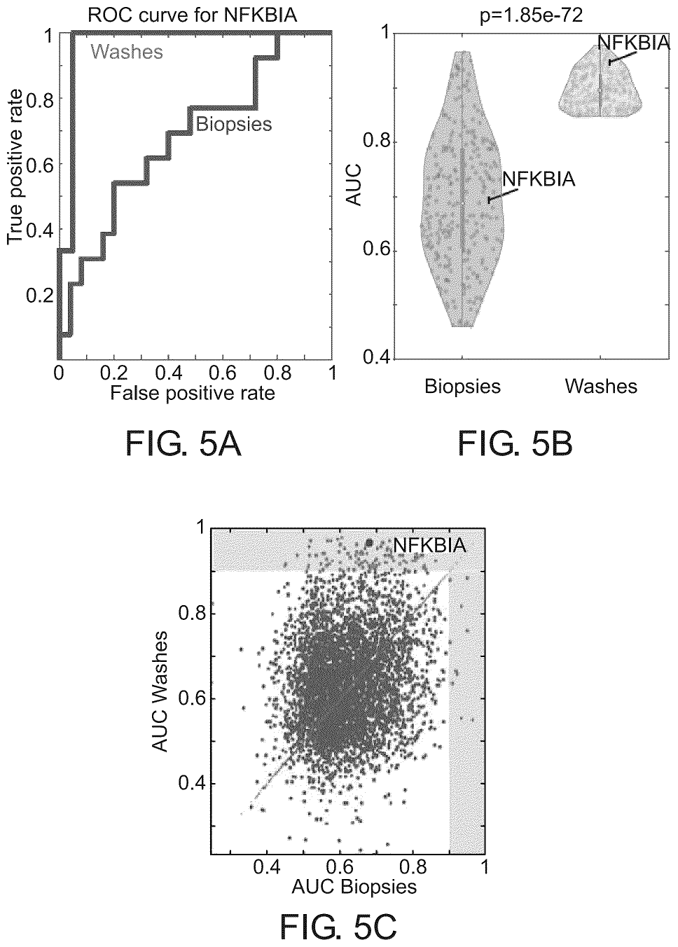

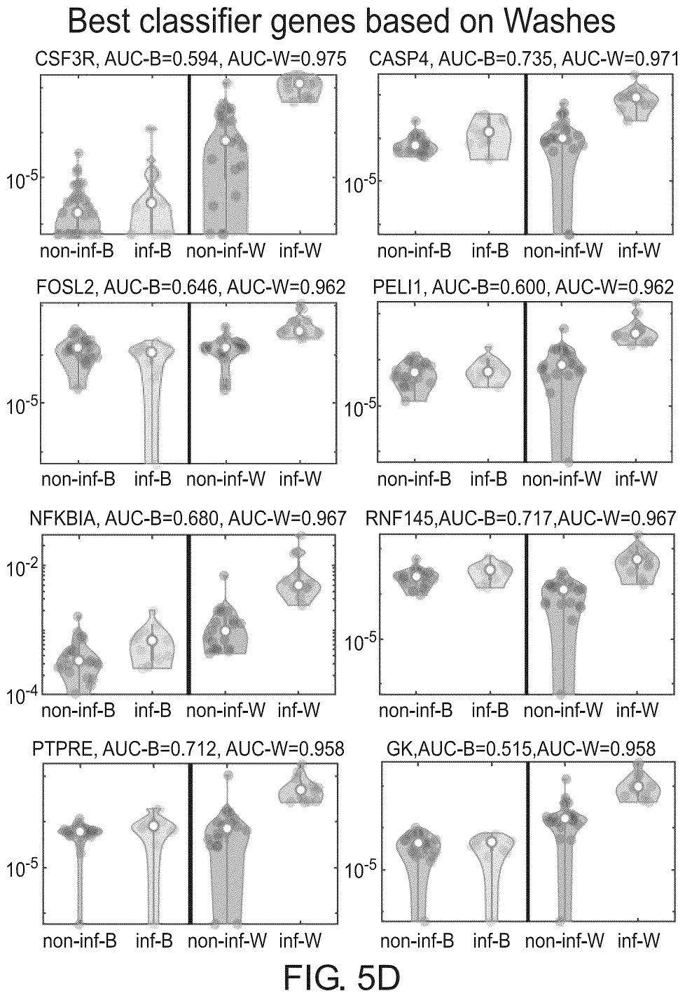

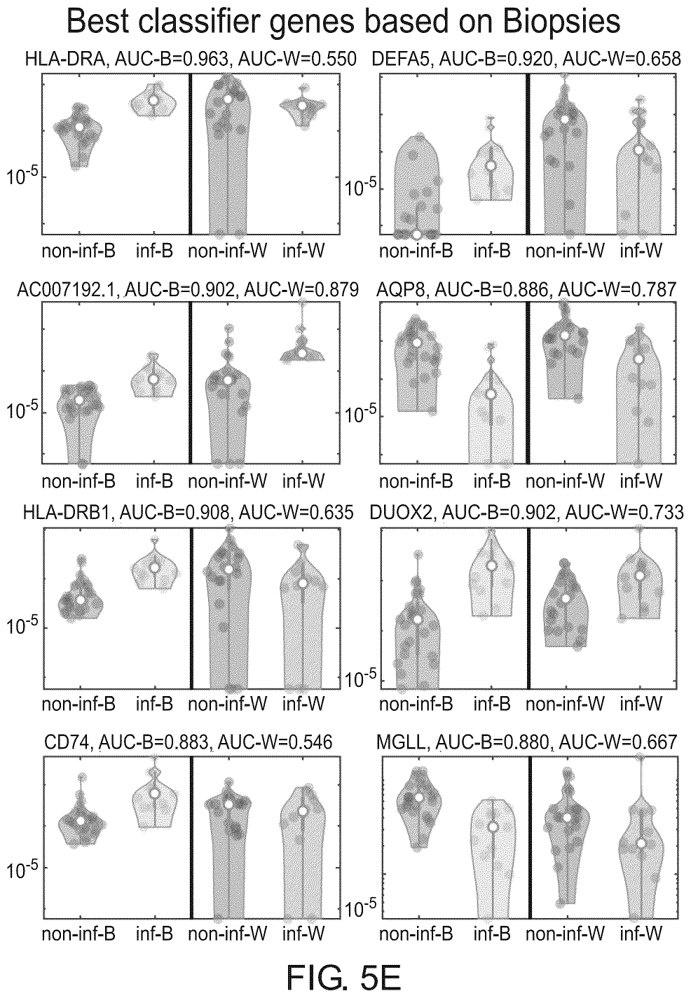

The present inventors have now examined whether host transcriptomics of fecal samples could serve as a diagnostic tool for IBD patients. Specifically, the present inventors sequenced the RNA of biopsies and fecal-wash samples from IBD patients and controls undergoing lower endoscopy. The present inventors showed that the host fecal-transcriptome carried information that was distinct from biopsy RNAseq and fecal proteomics. Transcriptomics of fecal washes, yet not of biopsies, from patients with histological inflammation were significantly correlated to one another (p=5.3*10-12), as illustrated in FIGS. 2A-E. Fecal-transcriptome was significantly more powerful in identifying histological inflammation compared to intestinal biopsies (150 genes with area-under-the-curve >0.9 in fecal samples versus 10 genes in biopsy RNAseq), as illustrated in FIGS. 5A-E.

The present inventors thus deduce that fecal wash host transcriptome is a powerful non-invasive biomarker reflecting histological inflammation, opening the way to the identification of important correlates and therapeutic targets that may be obscured using biopsy transcriptomics. Since the fecal wash host transcriptome was shown to be informative on the state of histological inflammation in the gastrointestinal tract, the present inventors propose that RNA transcriptome analysis of fecal samples themselves can also serve as a diagnostic tool for IBD.

According to one aspect of the present invention a method is provided for diagnosing an inflammatory bowel disease (IBD) of a subject comprising analyzing the RNA expression level of at least one human gene in a fecal RNA sample of the subject, wherein when the expression level of a human gene set forth in Table 1 is statistically significantly altered (e.g. increased) over the level of the gene in a fecal RNA sample of a control subject, it is indicative of the inflammatory bowel disease.

According to another aspect of the present invention there is provided a method of diagnosing an inflammatory bowel disease (IBD) of a subject comprising analyzing the RNA expression level of at least one human gene in a fecal RNA sample of the subject, wherein the gene is selected from the group consisting of CSF3R, CASP4, NFKB1A, RNF145, FOSL2, PEL1, RTPRE, GK, MX2, NAGK, MCTP2, SLCO3A1, STAT1, RASSF3, MARCKS, SAT1, NFKBIA, VPS37B. RNF149, HLA-E, PLAUR, MSN, HIF1A and NBPF14, wherein when the expression level is above a predetermined amount it is indicative of the inflammatory bowel disease, thereby diagnosing the inflammatory bowel disease.

Inflammatory bowel diseases (IBD) are severe gastrointestinal disorders characterized by intestinal inflammation and tissue remodeling, that increase in frequency and may prove disabling for patients. The major forms of IBD, ulcerative colitis (UC) and Crohn's disease are chronic, relapsing conditions that are clinically characterized by abdominal pain, diarrhea, rectal bleeding, and fever.

As used herein, the term “diagnosing” refers to determining presence or absence of the disease, classifying the disease (e.g. classifying the disease according to the histological inflammation status), determining a severity of the disease, monitoring disease progression, forecasting an outcome of a pathology and/or prospects of recovery and/or screening of a subject for the inflammatory bowel disease.

According to a specific embodiment, the diagnosing refers to determining if the subject is in remission from the disease.

In another embodiment, the diagnosing comprises determining if the subject is suitable for a particular treatment. Thus, for example if the RNA determinants indicate an increase in inflammation, an anti-inflammatory drug (e.g. anti-TNF-α therapy).

The RNA sample may be derived from solid feces (i.e. stool sample) or a fecal wash (as described herein below). The RNA may comprise total RNA, mRNA, mitochondrial RNA, chloroplast RNA, DNA-RNA hybrids, viral RNA, cell free RNA, and mixtures thereof. In one embodiment, the RNA sample is substantially devoid of DNA. In another embodiment, the RNA sample is substantially devoid of protein.

The sample may be fresh or frozen.

Isolation, extraction or derivation of RNA may be carried out by any suitable method. Isolating RNA from a biological sample generally includes treating a biological sample in such a manner that the RNA present in the sample is extracted and made available for analysis. Any isolation method that results in extracted RNA may be used in the practice of the present invention. It will be understood that the particular method used to extract RNA will depend on the nature of the source.

Methods of RNA extraction are well-known in the art and further described herein under.

Phenol based extraction methods: These single-step RNA isolation methods based on Guanidine isothiocyanate (GITC)/phenol/chloroform extraction require much less time than traditional methods (e.g. CsCl₂ ultracentrifugation). Many commercial reagents (e.g. Trizol, RNAzol, RNAWIZ) are based on this principle. The entire procedure can be completed within an hour to produce high yields of total RNA.

Silica gel—based purification methods: RNeasy is a purification kit marketed by Qiagen. It uses a silica gel-based membrane in a spin-column to selectively bind RNA larger than 200 bases. The method is quick and does not involve the use of phenol.

Oligo-dT based affinity purification of mRNA: Due to the low abundance of mRNA in the total pool of cellular RNA, reducing the amount of rRNA and tRNA in a total RNA preparation greatly increases the relative amount of mRNA. The use of oligo-dT affinity chromatography to selectively enrich poly (A)+RNA has been practiced for over 20 years. The result of the preparation is an enriched mRNA population that has minimal rRNA or other small RNA contamination. mRNA enrichment is essential for construction of cDNA libraries and other applications where intact mRNA is highly desirable. The original method utilized oligo-dT conjugated resin column chromatography and can be time consuming. Recently more convenient formats such as spin-column and magnetic bead based reagent kits have become available.

The sample may also be processed prior to carrying out the diagnostic methods of the present invention. Processing of the sample may involve one or more of: filtration, distillation, centrifugation, extraction, concentration, dilution, purification, inactivation of interfering components, addition of reagents, and the like.

The present inventors contemplate negative genomic selection of abundant microbial transcripts such as bacterial (SEQ ID NOs: 1-20) and/or fungal rRNA (SEQ ID NOs: 21-24) prior to the analysis. This increases the fraction of human exonic reads in the sequenced samples. This may be effected on the solid fecal samples or on fecal wash samples.

Examples of additional RNA transcripts that may be depleted include, but are not limited to Eubacterium rectale, Faccalibacterium prausnitzii, Bifidobacterium adolescentis, Ruminococcus sp 5 1 39BFAA, Bifidobacterium longum, Subdoligranulum, Ruminococcus gnavus, Escherichia coli, Ruminococcus torques, Akkermansia muciniphila, Ruminococcus bromii, Dialister invisus, Collinsella acrofaciens, Bacteroides uniformis, Bacteroides vulgatus, Eubacterium hallii, Dorea longicatena, Prevotella copri, Alistipes putredinis and Bifidobacterium bifidum.

The present inventors contemplate depletion of at least one, at least two, at least three, at least four or at least 5 of the above identified bacteria.

Methods of depleting particular RNAs are known in the art. For example DNA probes may be synthesized to be reverse-complement to the bacterial or fungal transcripts. Next, RNase H enzyme may be used which digests RNA-DNA specific hybrids. This leads to the selective digestion of only RNA molecules targeted by the DNA probes. Lastly, endocucleases such as DNase I enzyme may be used to remove the left over DNA probes and other DNA residues left in the sample after RNA extraction. Another method for depleting particular RNAs is by using nucleic acid probes (which are attached to an affinity tag) that specifically hybridize to the RNAs. Exemplary affinity tags include, but are not limited to hemagglutinin (HA), AviTag™M, V5, Myc. T7, FLAG, HSV. VSV-G, His, biotin, or streptavidin

After obtaining the RNA sample, cDNA may be generated therefrom. For synthesis of cDNA, template mRNA may be obtained directly from lysed cells or may be purified from a total RNA or mRNA sample. The total RNA sample may be subjected to a force to encourage shearing of the RNA molecules such that the average size of each of the RNA molecules is between 100-300 nucleotides, e.g. about 200 nucleotides. To separate the heterogeneous population of mRNA from the majority of the RNA found in the cell, various technologies may be used which are based on the use of oligo(dT) oligonucleotides attached to a solid support. Examples of such oligo(dT) oligonucleotides include: oligo(dT) cellulose/spin columns, oligo(dT)/magnetic beads, and oligo(dT) oligonucleotide coated plates.

According to another embodiment, long-read transcriptome sequencing is carried out, wherein the full length RNA molecule is sequenced (i.e. from the 3′polyA tail to the 5′ cap).

Generation of single stranded DNA from RNA requires synthesis of an intermediate RNA-DNA hybrid. For this, a primer is required that hybridizes to the 3′ end of the RNA. Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

According to a specific embodiment, the primer comprises a polydT oligonucleotide sequence.

Preferably the polydT sequence comprises at least 5 nucleotides. According to another is between about 5 to 50 nucleotides, more preferably between about 5-25 nucleotides, and even more preferably between about 12 to 14 nucleotides.

Following annealing of the primer (e.g. polydT primer) to the RNA sample, an RNA-DNA hybrid is synthesized by reverse transcription using an RNA-dependent DNA polymerase. Suitable RNA-dependent DNA polymerases for use in the methods and compositions of the invention include reverse transcriptases (RTs). Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus

(AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules.

Additional components required in a reverse transcription reaction include dNTPS (dATP, dCTP, dGTP and dTTP) and optionally a reducing agent such as Dithiothreitol (DTT) and MnCl₂.

Following cDNA synthesis, the present inventors contemplate amplifying the cDNA (e.g using a polymerase chain reaction—PCR, details of which are known in the art).

As mentioned, in order to diagnose IBD, the quantity of at least one, at least two, at least three, at least four, at least five, at least six, at least seven, at least eight, at least nine, at least ten human RNA determinant of Table 1, 5 or 6 is analyzed. According to another embodiment, no more than 20 RNA, 30, 40 or 50 RNA determinants set forth in Table 1 are analyzed in fecal washes or solid feces of a subject.

In another embodiment, in order to diagnose IBD, the quantity of at least one, at least two, at least three, at least four, at least five, at least six, at least seven, at least eight, at least nine, at least ten human RNA determinant of Table 5 or 6 is analyzed. Particularly relevant RNAs for diagnosing IBD from a solid fecal sample include CXCR1, CSF2RA, CLEC2B, GBP5, IL1B, FZD3, MMP25 and OSM. According to another embodiment, no more than 20 RNA, 30, 40 or 50 RNA determinants set forth in Tables 5 or 6 are analyzed in feces of a subject.

TABLE 1

| | | |
|---|---|---|
| 1 | MX2 | NM_002463 |
| 2 | CSF3R | NM_000760, NM_156038, NM_156039, NM_172313 |
| 3 | CD93 | NM_012072 |
| 4 | NAGK | NM_001330425, NM_001330426, NM_001365466, NM_017567 |
| 5 | MCTP2 | NM_001159643, NM_001159644, NM_018349 |
| 6 | SLCO3A1 | NM_001145044, NM_013272 |
| 7 | CASP4 | NM_001225, NM_033306, NM_033307 |
| 8 | NFKBIA | NM_020529 |
| 9 | STAT1 | NM_007315, NM_139266 |
| 10 | TLR4 | NM_003266, NM_138554, NM_138556, NM_138557 |
| 11 | RNF145 | NM_001199380, NM_001199381, NM_001199382, NM_001199383, NM_144726 |
| 12 | TECPR2 | NM_001172631, NM_014844 |
| 13 | KCNJ2 | NM_000891 |
| 14 | FAM49B | NM_001256763, NM_001330612, NM_001353242, NM_001353243, |
| | | NM_001353308 |
| 15 | PELI1 | NM_020651 |
| 16 | AKNA | NM_001317950, NM_001317952, NM_030767 |
| 17 | PTPRE | NM_001316676, NM_001316677, NM_001323354, NM_001323355, NM_001323356, |
| | | NM_001323357, NM_006504, NM_130435 |
| 18 | CLEC4E | NM_014358 |
| 19 | GK | NM_000167, NM_001128127, NM_001205019, NM_203391 |
| 20 | IL1R2 | NM_001261419, NM_004633, NM_173343 |
| 21 | ITGAX | NM_000887, NM_001286375 |
| 22 | MYO1F | NM_001348355, NM_012335 |
| 23 | LRRK2 | NM_198578 |
| 24 | LILRB3 | NM_001081450, NM_001320960, NM_006864 |
| 25 | FGR | NM_001042729, NM_001042747, NM_005248 |
| 26 | TYMP | NM_001113755, NM_001113756, NM_001257988, NM_001257989, NM_001953 |
| 27 | SH3BP5 | NM_001018009, NM_004844 |
| 28 | ZNF267 | NM_001265588, NM_003414 |
| 29 | RNF24 | NM_001134337, NM_001134338, NM_001321749, NM_007219 |
| 30 | AQP9 | NM_001320635, NM_001320636, NM_020980 |
| 31 | BCL6 | NM_001130845, NM_001134738, NM_001706, NM_138931 |
| 32 | FFAR2 | NM_001370087, NM_005306 |
| 33 | RNF144B | NM_182757 |
| 34 | RILPL2 | NM_145058 |
| 35 | SOCS3 | NM_003955 |
| 36 | ZDHHC18 | NM_032283 |
| 37 | PLAUR | NM_001005376, NM_001005377, NM_001301037, NM_002659 |
| 38 | TNFRSF1B | NM_001066 |
| 39 | HIF1A | NM_001243084, NM_001530, NM_181054 |
| 40 | ADAM8 | NM_001109, NM_001164489, NM_001164490 |
| 41 | ACSL1 | NM_001286708, NM_001286710, NM_001286711, NM_001286712, NM_001995 |
| 42 | PROK2 | NM_001126128, NM_021935 |
| 43 | NFKBID | NM_001321831, NM_001365705, NM_001365706, NM_032721, NM_139239 |
| 44 | FCAR | NM_002000, NM_133269, NM_133271, NM_133272, NM_133273, NM_133274, |
| | | NM_133277, NM_133278, NM_133279, NM_133280 |
| 45 | OSM | NM_001319108, NM_020530 |
| 46 | MR1 | NM_001194999, NM_001195000, NM_001195035, NM_001310213, NM_001531 |
| 47 | CD44 | NM_000610, NM_001001389, NM_001001390, NM_001001391, NM_001001392, |
| | | NM_001202555, NM_001202556, NM_001202557 |
| 48 | FYB1 | NM_001243093, NM_001349333, NM_001465, NM_018594, NM_199335 |
| 49 | TNFAIP3 | NM_001270507, NM_001270508, NM_006290 |
| 50 | TNFSF14 | NM_003807, NM_172014 |
| 51 | KDM6B | NM_001080424, NM_001348716 |
| 52 | MINDY1 | NM_001040217, NM_001163258, NM_001163259, NM_001163260, NM_001319998, |
| | | NM_018379 |
| 53 | PPP1R18 | NM_001134870, NM_133471 |
| 54 | CCR1 | NM_001295 |
| 55 | BASP1 | NM_001271606, NM_006317 |
| 56 | NBPF14 | NM_015383 |
| 57 | PLEKHO1 | NM_001304722, NM_001304723, NM_001304724, NM_016274 |
| 58 | ZEB2 | NM_001171653, NM_014795 |
| 59 | HCLS1 | NM_001292041, NM_005335 |
| 60 | PLIN5 | NM_001013706 |
| 61 | C5AR2 | NM_001271749, NM_001271750, NM_018485 |
| 62 | LCP2 | NM_005565 |
| 63 | KATNBL1 | NM_024713 |
| 64 | IGSF6 | NM_005849 |
| 65 | ABCA1 | NM_005502 |
| 66 | RHOH | NM_001278359, NM_001278360, NM_001278361, NM_001278362, NM_001278363, |
| | | NM_001278364, NM_001278365, NM_001278366, NM_001278367, NM_001278368, |
| | | NM_001278369, NM_004310 |
| 67 | LIMK2 | NM_001031801, NM_005569, NM_016733 |
| 68 | HCAR2 | NM_177551 |
| 69 | C5AR1 | NM_001736 |
| 70 | MAP3K3 | NM_001330431, NM_001363768, NM_002401, NM_203351 |
| 71 | TREM1 | NM_001242589, NM_001242590, NM_018643 |
| 72 | CSNK1G2 | NM_001319 |
| 73 | LYVE1 | NM_006691 |
| 74 | FCGR2A | NM_001136219, NM_021642 |
| 75 | TNFAIP2 | NM_001371220, NM_001371221, NM_006291 |
| 76 | RIPOR2 | NM_001286445, NM_001286446, NM_001286447, NM_001346031, NM_001346032, |
| | | NM_014722, NM_015864 |
| 77 | PHACTR1 | NM_001242648, NM_001322308, NM_001322309, NM_001322310, NM_001322311, |
| | | NM_001322312, NM_001322313, NM_001322314, NM_030948 |
| 78 | PLEK | NM_002664 |
| 79 | G0S2 | NM_015714 |
| 80 | MSN | NM_002444 |
| 81 | SLC45A4 | NM_001080431, NM_001286646, NM_001286648 |
| 82 | UNC13D | NM_199242 |
| 83 | NAMPT | NM_005746, NM_182790 |
| 84 | SAT1 | NM_002970 |
| 85 | ENTPD1 | NM_001098175, NM_001164178, NM_001164179, NM_001164181, NM_001164182, |
| | | NM_001164183, NM_001312654, NM_001320916, NM_001776 |
| 86 | ARL11 | NM_138450 |
| 87 | S100A12 | NM_005621 |
| 88 | FPR1 | NM_001193306, NM_002029 |
| 89 | DDX60L | NM_001012967, NM_001291510, NM_001345927 |
| 90 | MKNK1 | NM_001135553, NM_003684, NM_198973 |
| 91 | IL1RN | NM_000577, NM_001318914, NM_173841, NM_173842, NM_173843 |
| 92 | VPS37B | NM_024667 |
| 93 | FMNL1 | NM_005892 |
| 94 | DOCK8 | NM_001190458, NM_001193536, NM_203447 |
| 95 | IL1B | NM_000576 |
| 96 | DYSF | NM_001130455, NM_001130976, NM_001130977, NM_001130978, NM_001130979, |
| | | NM_001130980, NM_001130981, NM_001130982, NM_001130983, NM_001130984, |
| | | NM_001130985, NM_001130986, NM_001130987, NM_003494 |
| 97 | SELL | NM_000655 |
| 98 | CNN2 | NM_001303499, NM_001303501, NM_004368, NM_201277 |
| 99 | ARHGAP15 | NM_018460 |
| 100 | GBP1 | NM_002053 |
| 101 | CREB5 | NM_001011666, NM_004904, NM_182898, NM_182899 |
| 102 | PFKFB3 | NM_001145443, NM_001282630, NM_001314063, NM_001323016, NM_001323017, |
| | | NM_001363545, NM_004566 |
| 103 | GLIPR2 | NM_001287010, NM_001287011, NM_001287012, NM_001287013, NM_001287014, |
| | | NM_022343 |
| 104 | DOK3 | NM_001144875, NM_001144876, NM_001308235, NM_001308236, NM_024872 |
| 105 | TRIM22 | NM_001199573, NM_006074 |
| 106 | IFI16 | NM_001206567, NM_001364867, NM_005531 |
| 107 | MYADM | NM_001020818, NM_001020819, NM_001020820, NM_001020821, NM_001290188, |
| | | NM_001290189, NM_001290190, NM_001290191, NM_001290192, NM_001290193, |
| | | NM_001290194, NM_138373 |
| 108 | KLF2 | NM_016270 |
| 109 | CELF2 | NM_001025076, NM_001025077, NM_001083591, NM_001326317, NM_001326318, |
| | | NM_001326319, NM_001326320, NM_001326321, NM_001326323, NM_001326324, |
| | | NM_001326325, NM_001326326, NM_001326327, NM_001326328, NM_001326329, |
| | | NM_001326330, NM_001326331, NM_001326332, NM_001326333, NM_001326334, |
| | | NM_001326335, NM_001326336, NM_001326337, NM_001326338, NM_001326339, |
| | | NM_001326340, NM_001326341, NM_001326342, NM_001326343, NM_001326344, |
| | | NM_001326345, NM_001326346, NM_001326347, NM_001326348, NM_001326349, |
| | | NM_006561 |
| 110 | ACTN1 | NM_001102, NM_001130004, NM_001130005 |
| 111 | ICAM1 | NM_000201 |
| 112 | IRAK3 | NM_001142523, NM_007199 |
| 113 | LCP1 | NM_002298 |
| 114 | PADI4 | NM_012387 |
| 115 | S100A9 | NM_002965 |
| 116 | PTAFR | NM_000952, NM_001164721, NM_001164722, NM_001164723 |
| 117 | CXCR2 | NM_001168298, NM_001557 |
| 118 | IL1RAP | NM_001167928, NM_001167929, NM_001167930, NM_001167931, NM_001364879, |
| | | NM_001364880, NM_001364881, NM_002182, NM_134470 |
| 119 | DENND3 | NM_001352890, NM_001352891, NM_001362798, NM_014957 |
| 120 | ANKRD44 | NM_001195144, NM_001367495, NM_001367496, NM_001367497, NM_153697 |
| 121 | CYTH4 | NM_001318024, NM_013385 |
| 122 | INHBA | NM_002192 |
| 123 | CSRNP1 | NM_001320559, NM_001320560, NM_033027 |
| 124 | CLEC7A | NM_022570, NM_197947, NM_197948, NM_197949, NM_197950, NM_197951, |
| | | NM_197952, NM_197953, NM_197954 |
| 125 | MCTP1 | NM_001002796, NM_001297777, NM_024717 |
| 126 | SLC11A1 | NM_000578, NM_001032220 |
| 127 | DDX21 | NM_001256910, NM_004728 |
| 128 | TRIB1 | NM_001282985, NM_025195 |
| 129 | CCL3 | NM_002983 |
| 130 | TLR1 | NM_003263 |
| 131 | PIM3 | NM_001001852 |
| 132 | TLR2 | NM_001318787, NM_001318789, NM_001318790, NM_001318791, |
| | | NM_001318793, NM_001318795, NM_001318796, NM_003264 |
| 133 | CXCL3 | NM_002090 |
| 134 | PLK3 | NM_004073 |
| 135 | NLRP1 | NM_001033053, NM_014922, NM_033004, NM_033006, NM_033007 |
| 136 | UBR1 | NM_174916 |
| 137 | MOB3A | NM_130807 |
| 138 | PDE4B | NM_001037339, NM_001037340, NM_001037341, NM_001297440, |
| | | NM_001297441, NM_001297442, NM_002600 |
| 139 | PLAU | NM_001145031, NM_001319191, NM_002658 |
| 140 | RELT | NM_032871, NM_152222 |
| 141 | VNN2 | NM_001242350, NM_004665, NM_078488 |
| 142 | CDKN2D | NM_001800, NM_079421 |
| 143 | ADAM19 | NM_023038, NM_033274 |
| 144 | STAT5B | NM_012448 |
| 145 | RALGAPA2 | NM_020343 |
| 146 | BNIP2 | NM_001320674, NM_001320675, NM_001368057, NM_001368058, |
| | | NM_001368059, NM_001368060, NM_001368061, NM_004330 |
| 147 | CSF2RA | NM_001161529, NM_001161530, NM_001161531, NM_001161532, NM_006140, |
| | | NM_172245, NM_172246, NM_172247, NM_172248, NM_172249 |
| 148 | SCN1B | NM_001037, NM_001321605, NM_199037 |
| 149 | LMNB1 | NM_001198557, NM_005573 |
| 150 | IL7R | NM_002185 |
| 151 | PI3 | NM_002638 |
| 152 | SLC12A9 | NM_001267812, NM_001267814, NM_001363493, NM_001363494, NM_020246 |
| 153 | ST3GAL1 | NM_003033, NM_173344 |
| 154 | MMP9 | NM_004994 |
| 155 | GZF1 | NM_001317012, NM_001317019, NM_022482 |
| 156 | AGTPBP1 | NM_001286715, NM_001286717, NM_001330701, NM_015239 |
| 157 | SLA | NM_001045556, NM_001045557, NM_001282964, NM_001282965, NM_006748 |
| 158 | CD86 | NM_001206924, NM_001206925, NM_006889, NM_175862, NM_176892 |
| 159 | GBP5 | NM_001134486, NM_052942 |
| 160 | NFAM1 | NM_001318323, NM_001371362, NM_145912 |
| 161 | MEFV | NM_000243, NM_001198536 |
| 162 | KCNJ15 | NM_001276435, NM_001276436, NM_001276437, NM_001276438, |
| | | NM_001276439, NM_002243, NM_170736, NM_170737 |
| 163 | TNFAIP6 | NM_007115 |
| 164 | RBMS1 | NM_002897, NM_016836, NM_016839 |
| 165 | WDFY3 | NM_014991, NM_178583, NM_178585 |
| 166 | HES4 | NM_001142467, NM_021170 |
| 167 | IL18R1 | NM_001282399, NM_001371418, NM_001371419, NM_001371420, |
| | | NM_001371421, NM_001371422, NM_001371423, NM_001371424, NM_003855 |
| 168 | FPR2 | NM_001005738, NM_001462 |
| 169 | GNG2 | NM_001243773, NM_001243774, NM_053064 |
| 170 | FBRS | NM_001105079, NM_022452 |
| 171 | NFKB2 | NM_001077494, NM_001261403, NM_001288724, NM_001322934, NM_001322935, |
| | | NM_002502 |
| 172 | SNX10 | NM_001199835, NM_001199837, NM_001199838, NM_001318198, NM_001318199, |
| | | NM_001362753, NM_001362754, NM_013322 |
| 173 | CKLF | NM_001040138, NM_001040139, NM_016326, NM_016951, NM_181640, NM_181641 |
| 174 | ZNF200 | NM_001145446, NM_001145447, NM_001145448, NM_003454, NM_198087, |
| | | NM_198088 |
| 175 | VNN3 | NM_001291702, NM_001291703, NM_001368149, NM_001368150, NM_001368151, |
| | | NM_001368152, NM_001368154, NM_001368155, NM_001368156, NM_018399, |
| | | NM_078625 |
| 176 | DSE | NM_001080976, NM_001322937, NM_001322938, NM_001322939, NM_001322940, |
| | | NM_001322941, NM_001322943, NM_001322944, NM_013352 |
| 177 | PLCB2 | NM_001284297, NM_001284298, NM_001284299, NM_004573 |
| 178 | GYG1 | NM_001184720, NM_001184721, NM_004130 |
| 179 | ATG16L2 | NM_001318766, NM_033388 |
| 180 | TNFRSF10C | NM_003841 |
| 181 | PECAM1 | NM_000442 |
| 182 | NDE1 | NM_001143979, NM_017668 |
| 183 | CD69 | NM_001781 |
| 184 | CEP63 | NM_001042383, NM_001042384, NM_001042400, NM_001353108, NM_001353109, |
| | | NM_001353110, NM_001353111, NM_001353112, NM_001353113, NM_001353117, |
| | | NM_001353118, NM_001353119, NM_001353120, NM_001353121, NM_001353122, |
| | | NM_001353123, NM_001353124, NM_001353125, NM_001353126, NM_025180 |
| 185 | ARHGAP30 | NM_001025598, NM_001287600, NM_001287602, NM_181720 |
| 186 | S100A4 | NM_002961, NM_019554 |
| 187 | SCARF1 | NM_003693, NM_145350, NM_145352 |
| 188 | JAK3 | NM_000215 |
| 189 | FLOT2 | NM_001330170, NM_004475 |
| 190 | GLT1D1 | NM_001366886, NM_001366887, NM_001366888, NM_001366889, NM_144669 |
| 191 | HIP1 | NM_001243198, NM_005338 |
| 192 | HCK | NM_001172129, NM_001172130, NM_001172131, NM_001172132, NM_001172133, |
| | | NM_002110 |
| 193 | SELPLG | NM_001206609, NM_003006 |
| 194 | ARRB2 | NM_001257328, NM_001257329, NM_001257330, NM_001257331, NM_001330064, |
| | | NM_004313, NM_199004 |
| 195 | ZNF438 | NM_001143766, NM_001143767, NM_001143768, NM_001143769, NM_001143770, |
| | | NM_001143771, NM_182755 |
| 196 | LINC00694 | Ensembl: ENSG00000225873 |
| 197 | FAM129A | NP_443198.1 |
| 198 | AC007192.1 | ENSG00000268173 |
| 199 | AL512428.1 | ENSG00000282804 |
| 200 | AC069368.1 | ENSG00000249240 |

Particular combinations of RNAs contemplated by the present invention which may be analyzed are set forth below:

NFKBIA+CASP4; NFKBIA+CFLAR, NFKBIA+MX2, NFKBIA+STAT1, NFKBIA+GK, PELI1+CASP4, PELI1+CFLAR, PELI1+MX2, PELI1+STAT1, PELI1+GK, FAM49B+CASP4, FAM49B+CFLAR, FAM49B+MX2, FAM49B+STAT1, FAM49B+GK, CSF3R+CASP4, CSF3R+CFLAR, CSF3R+MX2, CSF3R+STAT1, CSF3R+CSF3R+GK, PTPRE+CASP4, PTPRE+CFLAR, PTPRE+MX2, PTPRE+STAT1 and PTPRE+GK.

More specifically, in order to diagnose IBD, the quantity of at least one human RNA determinant of Table 1, Table 5 or Table 6 is measured in RNA isolated from feces or a fecal wash of the subject. In another embodiment, at least two human RNA determinants of Table 1 are measured in RNA isolated from feces or a fecal wash of the subject. In another embodiment, at least three human RNA determinants of Table 1 are measured in RNA isolated from a solid fecal sample or a fecal wash of the subject.

In another embodiment, at least four human RNA determinants of Table 1 are measured in RNA isolated from feces or a fecal wash of the subject. In another embodiment, at least five human RNA determinants of Table 1, Table 5 or Table 6 are measured in RNA isolated from a solid fecal sample or a fecal wash of the subject.

According to particular embodiments, when the level of the RNA determinant in Table 1, Table 5 or Table 6 is above a predetermined level (e.g. above the level that is present in a control sample derived from a subject that does not have an inflammatory disease of the gut (e.g. a healthy subject); it is indicative that the subject has an inflammatory bowel disease (i.e. an inflammatory bowel disease may be ruled in). In another embodiment, when the level of the RNA determinant in Table 1 is above a predetermined level (e.g. above the level that is present in a control sample derived from a subject that does not have an inflammatory disease of the gut (e.g. a healthy subject); it is indicative of increased inflammation (e.g. corresponding to histological inflammation).

According to particular embodiments, when the level of the RNA determinant in Table 1, Table 5 or Table 6 is above a predetermined level (e.g. above the level that is present in a control sample derived from a previous sample of the subject), it is indicative that the inflammatory bowel disease has become more severe.

In one embodiment, when the level of RNA of one of the determinants in Table 1, Table 5 or Table 6 is at least 10% higher than the amount in the control sample, an IBD is ruled in.

In one embodiment, when the level of RNA of one of the determinants in Table 1, Table 5 or Table 6 is at least 20% higher than the amount in the control sample, an IBD is ruled in.

In one embodiment, when the level of RNA of one of the determinants in Table 1, Table 5 or Table 6 is at least 30% higher than the amount in the control sample, an IBD is ruled in.

In one embodiment, when the level of RNA of one of the determinants in Table 1, Table 5 or Table 6 is at least 40% higher than the amount in the control sample, an IBD is ruled in.

In one embodiment, when the level of RNA of one of the determinants in Table 1, Table 5 or Table 6 is at least 50% higher than the amount in the control sample, an IBD is ruled in.

In one embodiment, when the level of RNA of one of the determinants in Table 1, Table 5 or Table 6 is at least 100% higher than the amount in the control sample, an IBD is ruled in.

Alternatively or additionally, when the level of determinant in Table 2 is below a predetermined level, it is indicative that the subject does not have an inflammatory bowel disease.

According to a particular embodiment, the RNA determinant is set forth in Tables 2 or 3.

TABLE 2
Gene name

MARCKS
SAT1
NFKBIA
VPS37B
RNF149
HLA-E
PLAUR
MSN
HIF1A
NBPF14

TABLE 3
Gene name

MX2
CSF3R
NAGK
MCTP2
SLCO3A1
CASP4
NFKBIA
STAT1
RNF145
RASSF3

More specifically, in order to diagnose IBD, the quantity of at least one human RNA determinant of Table 2 is measured in RNA isolated from a solid fecal sample or a fecal wash of the subject. In another embodiment, at least two human RNA determinants of Table 2 are measured in RNA isolated from a solid fecal sample or a fecal wash of the subject. In another embodiment, at least three human RNA determinants of Table 2 are measured in RNA isolated from feces or a fecal wash of the subject.

In another embodiment, at least four human RNA determinants of Table 2 are measured in RNA isolated from a solid fecal sample or a fecal of the subject. In another embodiment, at least five human RNA determinants of Table 2 are measured in RNA isolated from feces or a fecal wash of the subject.

According to particular embodiments, when the level of the RNA determinant in Table 2 is above a predetermined level (e.g. above the level that is present in a control sample derived from a subject that does not have an inflammatory disease of the gut (e.g. a healthy subject); it is indicative that the subject has an inflammatory bowel disease (i.e. an inflammatory bowel disease may be ruled in). In another embodiment, when the level of the RNA determinant in Table 2 is above a predetermined level (e.g. above the level that is present in a control sample derived from a subject that does not have an inflammatory disease of the gut (e.g. a healthy subject); it is indicative of increased inflammation (e.g. corresponding to histological inflammation).

According to particular embodiments, when the level of the RNA determinant in Table 2 is above a predetermined level (e.g. above the level that is present in a control sample derived from a previous sample of the subject), it is indicative that the inflammatory bowel disease has become more severe.

In one embodiment, when the level of RNA of one of the determinants in Table 2 is at least 10% higher than the amount in the control sample, an IBD is ruled in.

In one embodiment, when the level of RNA of one of the determinants in Table 2 is at least 20% higher than the amount in the control sample, an IBD is ruled in.

In one embodiment, when the level of RNA of one of the determinants in Table 2 is at least 30% higher than the amount in the control sample, an IBD is ruled in.

In one embodiment, when the level of RNA of one of the determinants in Table 2 is at least 40% higher than the amount in the control sample, an IBD is ruled in.

In one embodiment, when the level of RNA of one of the determinants in Table 2 is at least 50% higher than the amount in the control sample, an IBD is ruled in.

In one embodiment, when the level of RNA of one of the determinants in Table 2 is at least 100% higher than the amount in the control sample, an IBD is ruled in.

Alternatively or additionally, when the level of determinant in Table 2 is below a predetermined level, it is indicative that the subject does not have an inflammatory bowel disease.

In order to diagnose IBD, the quantity of at least one human RNA determinant of Table 3 is measured in RNA isolated from a fecal wash of the subject. In another embodiment, in order to diagnose IBD, the quantity of at least two human RNA determinants of Table 3 are measured in RNA isolated from a fecal wash of the subject. In another embodiment, in order to diagnose IBD, the quantity of at least three human RNA determinants of Table 3 are measured in RNA isolated from a fecal wash of the subject. In another embodiment, in order to diagnose IBD, the quantity of at least four human RNA determinants of Table 3 are measured in RNA isolated from a fecal wash of the subject. In another embodiment, in order to diagnose IBD, the quantity of at least five human RNA determinants of Table 3 are measured in RNA isolated from a fecal wash of the subject.

According to particular embodiments, when the level of the RNA determinant in Table 3 is above a predetermined level (e.g. above the level that is present in a control sample derived from a subject that does not have an inflammatory disease of the gut (e.g. a healthy subject); it is indicative that the subject has an inflammatory bowel disease (i.e. an inflammatory bowel disease may be ruled in).

In another embodiment, when the level of the RNA determinant in Table 3 is above a predetermined level (e.g. above the level that is present in a control sample derived from a subject that does not have an inflammatory disease of the gut (e.g. a healthy subject); it is indicative of increased inflammation (e.g. corresponding to histological inflammation).

According to particular embodiments, when the level of the RNA determinant in Table 3 is above a predetermined level (e.g. above the level that is present in a control sample derived from a previous sample of the subject), it is indicative that the inflammatory bowel disease has become more severe.

In one embodiment, when the level of RNA of one of the determinants in Table 3 is at least 10% higher than the amount in the control sample, an IBD is ruled in.

In one embodiment, when the level of RNA of one of the determinants in Table 3 is at least 20% higher than the amount in the control sample, an IBD is ruled in.

In one embodiment, when the level of RNA of one of the determinants in Table 3 is at least 30% higher than the amount in the control sample, an IBD is ruled in.

In one embodiment, when the level of RNA of one of the determinants in Table 3 is at least 40% higher than the amount in the control sample, an IBD is ruled in.

In one embodiment, when the level of RNA of one of the determinants in Table 3 is at least 50% higher than the amount in the control sample, an IBD is ruled in.

In one embodiment, when the level of RNA of one of the determinants in Table 3 is at least 100% higher than the amount in the control sample, an IBD is ruled in.

The term “fecal wash” refers to fecal material which is removed from the body in a liquid state. In one embodiment, the fecal fluid is suctioned from the subject during colonoscopy or sigmoidoscopy or gastroscopy, including fluid suctioned from the small or large intestine. Fecal wash can also be obtained via rectal tube suctioning, with or without rectal irrigation. In another embodiment, the fecal wash refers to a liquid stool sample collected by the patient after consumption of a laxative.

Alternatively or additionally, when the level of determinant in Table 3 is below a predetermined level, it is indicative that the subject does not have an inflammatory bowel disease.

The predetermined level of any of the aspects of the present invention may be a reference value derived from population studies, including without limitation, such subjects having a known inflammatory bowel disease, subject having the same or similar age range, subjects in the same or similar ethnic group, or relative to the starting sample of a subject undergoing treatment for a disease. Such reference values can be derived from statistical analyses and/or risk prediction data of populations obtained from mathematical algorithms and computed indices of infection. Reference determinant indices can also be constructed and used using algorithms and other methods of statistical and structural classification.

It will be appreciated that the control sample is the same sample type as the sample being analyzed.

According to this aspect of the present invention, no more than 30 RNA determinants are used in order to diagnose the IBD, no more than 25 RNA determinants are used in order to diagnose the IBD, no more than 20 RNA determinants are used in order to diagnose the IBD, no more than 15 RNA determinants are used in order to diagnose the IBD, no more than 10 RNA determinants are used in order to diagnose the IBD, no more than 5 RNA determinants are used in order to diagnose the IBD, no more than 4 RNA determinants are used in order to diagnose the IBD, no more than 3 RNA determinants are used in order to diagnose the IBD, no more than 2 RNA determinants are used in order to diagnose the IBD.

Methods of analyzing the amount of RNA are known in the art and include Northern Blot analysis, RT-PCR analysis, RNA in situ hybridization stain, DNA microarray, DNA chips, oligonucleotide microarray, RNA sequencing and deep sequencing.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

In a particular embodiment, the RNA sequencing is effected at the single cell level.

It will be appreciated that in order to analyze the amount of an RNA, oligonucleotides may be used that are capable of hybridizing thereto or to cDNA generated therefrom. According to one embodiment a single oligonucleotide is used to determine the presence of a particular determinant, at least two oligonucleotides are used to determine the presence of a particular determinant, at least five oligonucleotides are used to determine the presence of a particular determinant, at least four oligonucleotides are used to determine the presence of a particular determinant, at least five or more oligonucleotides are used to determine the presence of a particular determinant.

In one embodiment, the method of this aspect of the present invention is carried out using an isolated oligonucleotide which hybridizes to the RNA or cDNA of any of the determinants listed in Tables 1-2 by complementary base-pairing in a sequence specific manner, and discriminates the determinant sequence from other nucleic acid sequence in the sample. Oligonucleotides (e.g. DNA or RNA oligonucleotides) typically comprises a region of complementary nucleotide sequence that hybridizes under stringent conditions to at least about 8, 10, 13, 16, 18, 20, 22, 25, 30, 40, 50, 55, 60, 65, 70, 80, 90, 100, 120 (or any other number in-between) or more consecutive nucleotides in a target nucleic acid molecule. Depending on the particular assay, the consecutive nucleotides include the determinant nucleic acid sequence.

The term “isolated”, as used herein in reference to an oligonucleotide, means an oligonucleotide, which by virtue of its origin or manipulation, is separated from at least some of the components with which it is naturally associated or with which it is associated when initially obtained. By “isolated”, it is alternatively or additionally meant that the oligonucleotide of interest is produced or synthesized by the hand of man.

In order to identify an oligonucleotide specific for any of the determinant sequences, the gene/transcript of interest is typically examined using a computer algorithm which starts at the 5′ or at the 3′ end of the nucleotide sequence. Typical algorithms will then identify oligonucleotides of defined length that are unique to the gene, have a GC content within a range suitable for hybridization, lack predicted secondary structure that may interfere with hybridization, and/or possess other desired characteristics or that lack other undesired characteristics.

Following identification of the oligonucleotide it may be tested for specificity towards the determinant under wet or dry conditions. Thus, for example, in the case where the oligonucleotide is a primer, the primer may be tested for its ability to amplify a sequence of the determinant using PCR to generate a detectable product and for its non ability to amplify other determinants in the sample. The products of the PCR reaction may be analyzed on a gel and verified according to presence and/or size.

Additionally, or alternatively, the sequence of the oligonucleotide may be analyzed by computer analysis to see if it is homologous (or is capable of hybridizing to) other known sequences. A BLAST 2.2.10 (Basic Local Alignment Search Tool) analysis may be performed on the chosen oligonucleotide (worldwidewebdotncbidotnlmdotnihdotgov/blast/). The BLAST program finds regions of local similarity between sequences. It compares nucleotide or protein sequences to sequence databases and calculates the statistical significance of matches thereby providing valuable information about the possible identity and integrity of the ‘query’ sequences.

According to one embodiment, the oligonucleotide is a probe. As used herein, the term “probe” refers to an oligonucleotide which hybridizes to the determinant specific nucleic acid sequence to provide a detectable signal under experimental conditions and which does not hybridize to additional determinant sequences to provide a detectable signal under identical experimental conditions.

The probes of this embodiment of this aspect of the present invention may be, for example, affixed to a solid support (e.g., arrays or beads).

According to particular embodiments, the array does not comprise nucleic acids that specifically bind to more than 50 determinants, more than 40 determinants, 30 determinants, 20 determinants, 15 determinants, 10 determinants, 5 determinants or even 3 determinants.

Methods for immobilization of oligonucleotides to solid-state substrates are well established. Oligonucleotides, including address probes and detection probes, can be coupled to substrates using established coupling methods.

According to another embodiment, the oligonucleotide is a primer of a primer pair. As used herein, the term “primer” refers to an oligonucleotide which acts as a point of initiation of a template-directed synthesis using methods such as PCR (polymerase chain reaction) or LCR (ligase chain reaction) under appropriate conditions (e.g., in the presence of four different nucleotide triphosphates and a polymerization agent, such as DNA polymerase, RNA polymerase or reverse-transcriptase, DNA ligase, etc, in an appropriate buffer solution containing any necessary co-factors and at suitable temperature(s)). Such a template directed synthesis is also called “primer extension”. For example, a primer pair may be designed to amplify a region of DNA using PCR. Such a pair will include a “forward primer” and a “reverse primer” that hybridize to complementary strands of a DNA molecule and that delimit a region to be synthesized/amplified. A primer of this aspect of the present invention is capable of amplifying, together with its pair (e.g. by PCR) a determinant specific nucleic acid sequence to provide a detectable signal under experimental conditions and which does not amplify other determinant nucleic acid sequence to provide a detectable signal under identical experimental conditions.

According to additional embodiments, the oligonucleotide is about 8, 9, 10, 11, 12, 13, 14, 15, 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. It will be appreciated that when attached to a solid support, the probe may be of about 30-70, 75, 80, 90, 100, or more nucleotides in length.

The oligonucleotide of this aspect of the present invention need not reflect the exact sequence of the determinant nucleic acid sequence (i.e. need not be fully complementary), but must be sufficiently complementary to hybridize with the determinant nucleic acid sequence under the particular experimental conditions. Accordingly, the sequence of the oligonucleotide typically has at least 70% homology, preferably at least 80%, 90%, 95%, 97%, 99% or 100% homology, for example over a region of at least 13 or more contiguous nucleotides with the target determinant nucleic acid sequence. The conditions are selected such that hybridization of the oligonucleotide to the determinant nucleic acid sequence is favored and hybridization to other determinant nucleic acid sequences is minimized.

By way of example, hybridization of short nucleic acids (below 200 bp in length, e.g. 13-50 bp in length) can be effected by the following hybridization protocols depending on the desired stringency; (i) hybridization solution of 6× SSC and 1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tm, final wash solution of 3 M TMACl, 0.01 M sodium phosphate (pH 6.8), 1 mM EDTA (pH 7.6), 0.5% SDS at 1-1.5° C. below the Tm (stringent hybridization conditions) (ii) hybridization solution of 6× SSC and 0.1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tm, final wash solution of 3 M TMACl, 0.01 M sodium phosphate (pH 6.8), 1 mM EDTA (pH 7.6), 0.5% SDS at 1-1.5° C. below the Tm, final wash solution of 6×SSC, and final wash at 22° C. (stringent to moderate hybridization conditions); and (iii) hybridization solution of 6× SSC and 1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature at 2.5-3° C. below the Tm and final wash solution of 6× SSC at 22° C. (moderate hybridization solution).

Oligonucleotides of the invention may be prepared by any of a variety of methods (see, for example, J. Sambrook et al., “Molecular Cloning: A Laboratory Manual”, 1989, 2.sup.nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5.sup.th Ed., John Wiley & Sons: Secaucus, N.J.). For example, oligonucleotides may be prepared using any of a variety of chemical techniques well-known in the art, including, for example, chemical synthesis and polymerization based on a template as described, for example, in S. A. Narang et al., Meth. Enzymol. 1979, 68: 90-98; E. L. Brown et al., Meth. Enzymol. 1979. 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066.

In certain embodiments, the detection probes or amplification primers or both probes and primers are labeled with a detectable agent or moiety before being used in amplification/detection assays. In certain embodiments, the detection probes are labeled with a detectable agent. Preferably, a detectable agent is selected such that it generates a signal which can be measured and whose intensity is related (e.g., proportional) to the amount of amplification products in the sample being analyzed.

The association between the oligonucleotide and detectable agent can be covalent or non-covalent. Labeled detection probes can be prepared by incorporation of or conjugation to a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

As shown in the Examples section herein below, the fecal wash of the sigmoid colon and the rectum was found to comprise exfoliated inflammatory cells of the gut, which is highly indicative of disease state (and more specifically diseases associated with inflammation).

Thus, according to another aspect of the present invention there is provided a method of diagnosing a disease of the gastrointestinal tract of a subject comprising analyzing the expression level of at least one gene in a fecal wash of the sigmoid colon or rectum of the subject, wherein the expression level is indicative of the disease of the gastrointestinal tract.

Diseases of the gastrointestinal tract include but are not limited to Irritable bowel syndrome (IBS), colon cancer, celiac disease and inflammatory bowel disease, including ulcerative colitis, Crohn's disease, microscopic colitis, Bechet's disease, immune-therapy-induced colitis or ileitis, cosinophilic gastritis/ileitis/colitis and collagenous gastritis/ileitis.

Exemplary genes which are informative on IBD which can be analyzed in fecal washes are provided in Table 2, herein above.

According to this aspect of the present invention, the analysis on the fecal wash samples may be carried out on the RNA level (as described herein above) or on the protein level (as described herein below).

Methods of measuring the levels of proteins are well known in the art and include, e.g., immunoassays based on antibodies to proteins, aptamers or molecular imprints.

The protein determinants can be detected in any suitable manner, but are typically detected by contacting a sample from the subject with an antibody, which binds the determinant and then detecting the presence or absence of a reaction product. The antibody may be monoclonal, polyclonal, chimeric, or a fragment of the foregoing, as discussed in detail above, and the step of detecting the reaction product may be carried out with any suitable immunoassay.

In one embodiment, the antibody which specifically binds the determinant is attached (either directly or indirectly) to a signal producing label, including but not limited to a radioactive label, an enzymatic label, a hapten, a reporter dye or a fluorescent label.

According to some embodiments of the invention, diagnosing of the subject for IBD is followed by substantiation of the screen results using gold standard methods.

In some embodiments, once a diagnosis has been obtained, screening for additional diseases may be recommended. For example routine colonoscopy may be recommended to monitor for colorectal cancer, since those with IBD are at a higher risk for developing it.

According to some embodiments of the invention, the method further comprises informing the subject of the diagnosis.

As used herein the phrase “informing the subject” refers to advising the subject that based on the diagnosis the subject should seek a suitable treatment regimen.

Once the diagnosis is determined, the results can be recorded in the subject's medical file, which may assist in selecting a treatment regimen and/or determining prognosis of the subject.

Optionally, once the diagnosis is confirmed using the methods described herein, the subject can be treated accordingly. IBD may be treated using anti-inflammatory drugs including, but not limited to corticosteroids (e.g. glucocorticoids such as budesonide (Uceris), prednisone (Prednisone Intensol, Rayos), prednisolone (Millipred, Prelone) and methylprednisolone (Medrol, Depo-Medrol)); 5-ASA drugs (aminosalicylates) including but not limited to balsalazide (Colazal), mesalamine (Apriso, Asacol HD, Canasa, Pentasa), olsalazine (Dipentum) and sulfasalazine (Azulfidine), immuno modulators including but not limited to methotrexate (Otrexup, Trexall, Rasuvo), azathioprinc (Azasan, Imuran) and mercaptopurine (Purixan).

Other agents suitable for treating IBD include inhibitors of TNF-alpha (including but not limited to adalimumab (Humira), golimumab (Simponi) and infliximab (Remicade). Other biologics for treating IBD include certolizumab (Cimzia); natalizumab (Tysabri); ustekinumab (Stelara) and vedolizumab (Entyvio).

Surgical interventions that can be recommended for treating IBD include strictureplasty to widen a narrowed bowel, closure or removal of fistulas, removal of affected portions of the intestines, for people with Crohn's disease and removal of the entire colon and rectum, for severe cases of UC.

The present inventors further conceive that the RNAs shown to be associated with the inflammatory status of the disease may be useful for selecting an agent for the treatment of an inflammatory bowel disease. This may be carried out as a method of selecting a known agent for a particular subject (i.e. personalized therapy) or as a more general method for uncovering novel drugs for the treatment of IBD.

Thus, according to another aspect of the present invention, a method of selecting an agent for the treatment of an inflammatory bowel disease (IBD) is provided. The method comprises;

(a) contacting the agent with an RNA sample derived from feces of a subject having the IBD; and

(b) analyzing the amount of at least one RNA set forth in Table 1, wherein a decrease in the amount of the at least one RNA in the presence of the agent as compared to the amount of the at least one RNA in the absence of the agent is indicative of an agent which is suitable for the treatment of the inflammatory bowel disease.

The RNA sample may be derived from solid feces of the subject or a liquid sample, as described herein above.

The contacting is typically carried out ex vivo.

Analysis of RNA is described herein above.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLE 1

### Materials and Methods

Patient population: The study groups included patients with ulcerative colitis or Crohn's colitis, or healthy controls. All control patients performed lower endoscopy for screening purposes and IBD patients underwent the procedure due to clinical indications (screening for dysplasia/assessment of disease status). Clinical and demographic parameters were obtained from patients' computerized files.

Sample collection: Upon endoscopy, biopsies (2 consecutive biopsies per patient—“double bite”) from the sigmoid colon were obtained and fecal fluid was suctioned from the sigmoid colon, at the beginning of the procedure before any through-the-scope washing was applied. Samples were snap-frozen in liquid nitrogen and stored at −80° C. until further analysis. In addition, stool samples were obtained from 4 patients (2 IBD patients and 2 controls) for proteomics analysis and stool calprotectin measurements.

Study Outcomes: The primary outcome was to map the transcriptomic profile of fecal washes in different patient groups (control, IBD with or without endoscopic and histological inflammation) and to identify biomarkers for classifying these groups. Secondary outcomes included a comparison of fecal washes to colonic biopsies and inference of the cellular composition of the fecal washes using computational deconvolution based on scRNAseq data.

Exclusion criteria
Patients younger than 18.
Undetermined diagnosis of UC or CD (IBD-unclassified).
Missing clinical/demographic data.
Patients with active endoscopic inflammation in the right colon only.

Biomarker measurements: Stool calprotectin was measured using a commercially available Calprosmart home-test19.

Definition of clinical remission: Clinical status was determined by HBI (Harvey-Bradshaw index) for Crohn's disease (CD) and by SCCAI (Simple Clinical Colitis Activity Index) for ulcerative colitis (UC) patients. Clinical remission was defined as HBI <5 for CD patients and SCCAI≤3 for UC patients²⁰⁻²¹.

Definition of mucosal healing and histological healing: Endoscopic and histological inflammation were graded according to standardized indices and by blinded gastroenterologists and pathologists, Endoscopic scores were determined prospectively during lower endoscopy. Mucosal healing was defined as absence of ulcers or lack of inflammation on endoscopic examination, for CD and UC respectively22. Histological inflammation was determined by a certified pathologist based on biopsies from the same sigmoid colon region used for the biopsy transcriptomics. Histological healing was retrospectively defined as grade 0 on the Nancy histological index.

RNA extraction: For colonic biopsies—snap frozen tissues (2 mm*2 mm) were thawed in 300 μl Tri-reagent and mechanically homogenized with bead beating, followed by a short centrifugation step to pull down beads and any tissue left-overs. For colonic washes—Tri-reagent was added at a ratio of 3:1, samples were allowed to thaw on ice followed by thorough mixing. A first centrifugation step was used (1 minute, 18,000 rpm) to eliminate fecal solids. Following this, ethanol was added in a ratio of 1:1 to the supernatant from the previous step and continued according to the manufacturer instructions of Direct-zol mini and micro prep kit (ZYMO research, R2052)²³.

RNA sequencing of samples: RNA was processed by the mcSCRBseq protocol²⁴ with minor modifications. RT reaction was applied on 10 ng of total RNA with a final volume of 10 μl (1× Maxima H Buffer, 1 mM dNTPs, 2 μM TSO* E5V6NEXT, 7.5% PEG8000, 20U Maxima H enzyme, 1 μl barcoded RT primer). Subsequent steps were applied as mentioned in the protocol. Library preparation was done using Nextera XT kit (Illumina) on 0.6 ng amplified cDNA. Library final concentration of 2 nM was loaded on NextSeq 500/550 (Illumina) sequencing machine aiming at 20 M reads per sample²³ with the following setting: Read1—16 bp, Index1—8 bp, Read2—66 bp.

Proteomic analysis: Fecal samples were lysed in lysis buffer containing 5% SDS, proteins were extracted, digested with trypsin, and tryptic peptides were subjected to LC-MS/MS analysis²⁵. Acquired raw data was analyzed using the MaxQuant software while searching against the human protein database, and downstream quantitative comparisons were calculated using the Perseus software²⁶.

Bioinformatics and computational analysis: Illumina output sequencing raw files were converted to FASTQ files using bcl2fastq package. To obtain the UMI counts, FASTQ files were aligned to the human reference genome (GRCh38.91) using zUMI package²⁷. Statistical analyses were performed with MATLAB R2018b. Mitochondrial genes and non-protein coding genes were removed from the analysis. Protein coding genes were extracted using the annotation in the Ensembl database (BioMart) for reference genome GRch38 version 91, using the R package “biomaRt” (version 2.44.4). Gene expression for each sample was consequently normalized by the sum of the UMIs of the remaining genes. Samples with less than 10,000 UMIs over the remaining genes were removed from the analyses. Clustering was performed with the MATLAB function clustergram over the Zscore-transformed expression matrix, using Spearman distances. Differential gene expression was performed using Wilcoxon ranksum tests and Benjamini-Hochberg FDR corrections. Computational deconvolution was performed using CIBERSORTx²⁸ using signature tables obtained from a single cell atlas of control and UC patients²⁹. Original cell type annotations were used, but subsequently coarse-grained into small number of cell types. M cells were removed from the analysis due to their low abundance. Receiver Operating Curve analyses were performed using the MATLAB function perfcurve. Gene Set Enrichment Analysis (GSEA)³⁰ was performed over the Hallmark and Kegg gene sets. Pathway analysis for the top-classifying fecal wash genes was performed using EnrichR³¹.

### Results

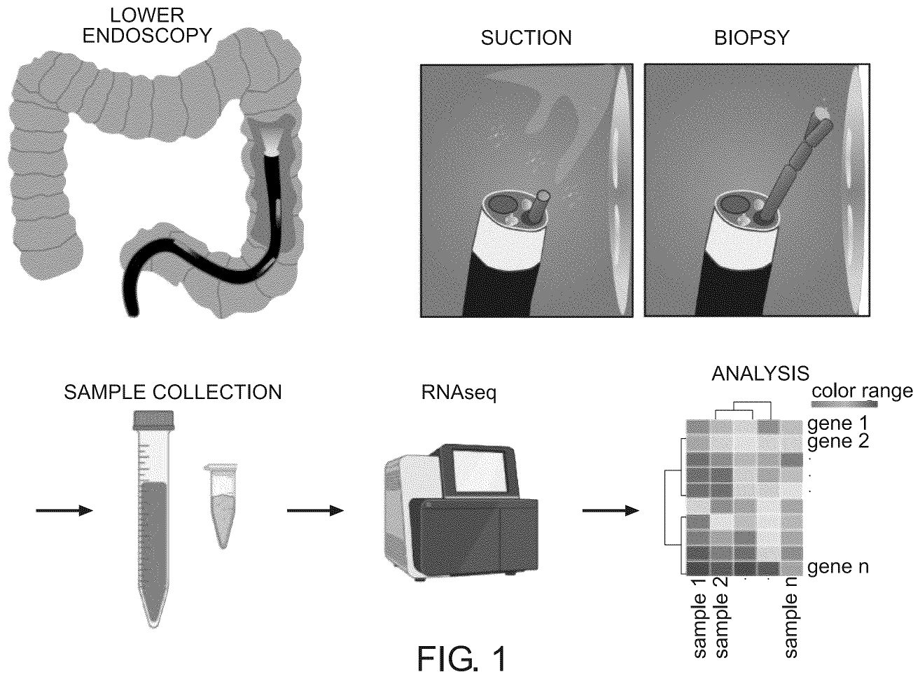

Cohort characteristics: In total, 39 biopsies and 39 matching fecal wash samples were obtained from 16 patients with ulcerative colitis, 3 patients with Crohn's colitis and 20 control subjects undergoing colonoscopy. Pairs of biopsies and matching washes were obtained concomitantly (FIG. 1, Table 4).

TABLE 4

| | | IBD | | Controls | | P value |
|---|---|---|---|---|---|---|
| N (%) | | 20 | (51) | 19 | (49) | |
| Age, years (median, IQR) | | 49 | (36-56) | 67 | (58-73) | 0.0008 |
| Female gender (%) | | 11 | (55) | 11 | (58) | 0.85 |
| Smoking at induction, n(%) | | 0 | (0) | 3 | (15) | 0.12 |
| Weight, kg -median(IQR) | | 80 | (69-87) | 68 | (63.6-79) | 0.9 |
| Concomitant medical | | 14 | (70) | 16 | (84) | 0.0015 |
| condition, n(%) | | | | | | |
| Disease duration, years- | | 14.5 | (4-31) | | | |
| median(IQR) | | | | | | |
| Previous surgery, n(%) | | 1 | (5) | | | |
| Concomitant 5-ASA | | 3 | (14) | | | |
| therapy, n(%) | | | | | | |
| Concomitant | | 1 | (5) | | | |
| immunomodulator | | | | | | |
| therapy, n(%) | | | | | | |
| Concomitant steroids, n(%) | | 5 | (24) | | | |
| Concomitant biological | | 10 | (48) | | | |
| therapy, n(%) | | | | | | |
| Disease | CD, Ileo- | 3 | (100)* | | | |
| location | colitis n(%) | | | | | |
| | UC, | 14 | (66) | | | |
| | pancolitis | | | | | |
| | n(%) | | | | | |
| | UC, left | 7 | (33) | | | |
| | sided colitis n(%) | | | | | |

| | | |
|---|---|---|
| Clinical remission at time | 9 | (47) |
| of endoscopy (median, IQR)* | | |
| Endoscopic remission at | 12 | (63) |
| time of endoscopy (median, | | |
| IQR)* | | |
| Histologic remission at | 7 | (37) |
| time of endoscopy (median, | | |
| IQR)* | | |

IBD—Inflammatory bowel disease, CD—Crohn's disease, UC—ulcerative colitis, IQR—interquartile range.
*Out of total CD patients (n = 3).
** Clinical remission was defined using the HBI and SCCAI scores for CD and UC respectively

Control patients were those undergoing lower endoscopy for screening purposes, recommended over the age of 50, and therefore they were significantly older than the IBD group (p<0.0008), with more comorbidities, other than IBD (p=0.0015, Table 1). Eleven (58%) of all IBD patients were treated with immunomodulator/biological therapy and five (26%) were on concomitant steroids at time of enrollment. Nine (47%) of the patients were in clinical remission, twelve (63%) were in endoscopic remission and seven (37%) achieved histologic remission as determined on the day of the lower endoscopy. Five fecal wash samples were excluded from the analysis due to technical dropouts.

Fecal wash host transcriptome is more informative than biopsy transcriptome in classifying patient disease status: Bulk RNA sequencing of all samples was performed using the UMI-based mcSCRBseq (see Methods) and the reads were mapped to the human genome. Gene expression signatures of colonic biopsies were found to be different from those of colonic washes (FIG. 2A, B). Biopsy samples with histological inflammation were not distinct from biopsy samples of patients without histological inflammation in the PCA or clustering analysis (FIG. 2C). In contrast, colonic fecal wash samples showed a clear separation between samples with and without histological inflammation (FIG. 2D).

The present inventors next sought to quantify the comparative ability of biopsy and fecal wash transcriptomics to inform on histological inflammation. To this end, they examined correlations between gene expression profiles of pairs of samples that both have histological inflammation compared to mixed pairs (one with and one without histological inflammation). There was no significant difference between transcriptomic profiles obtained from biopsies with histological inflammation compared to correlations between mixed biopsies (with or without histological inflammation) (p=0.98). However, fecal washes with histological inflammation were significantly more correlated to each other than mixed washes (FIG. 2E, p=5.3*10⁻¹²). This analysis therefore demonstrates that fecal wash transcriptomics may provide signatures for classifying patients with or without histological inflammation.

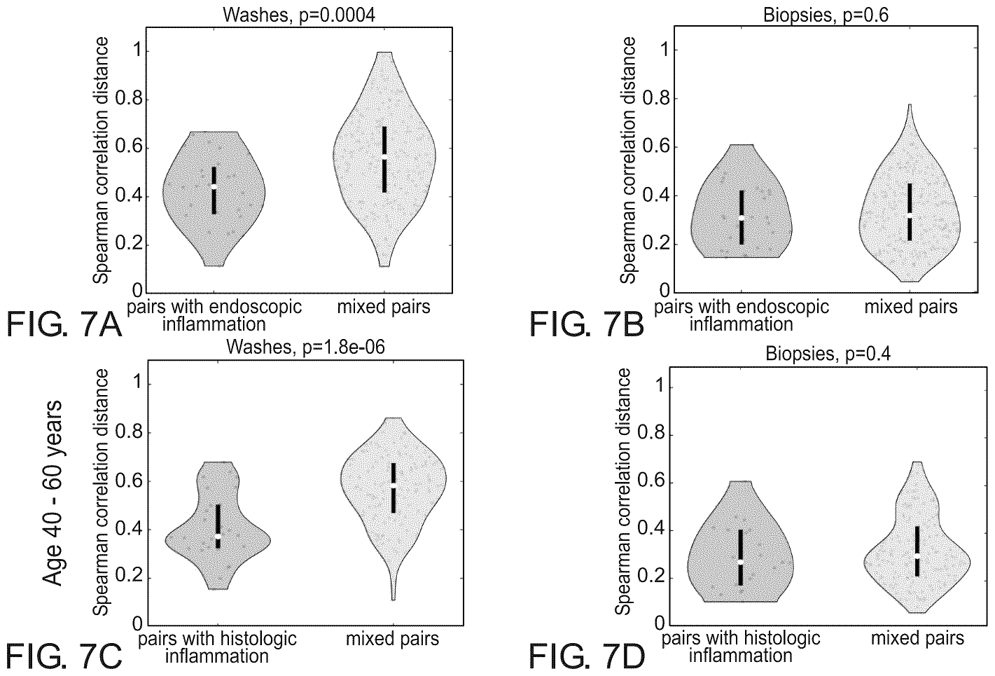

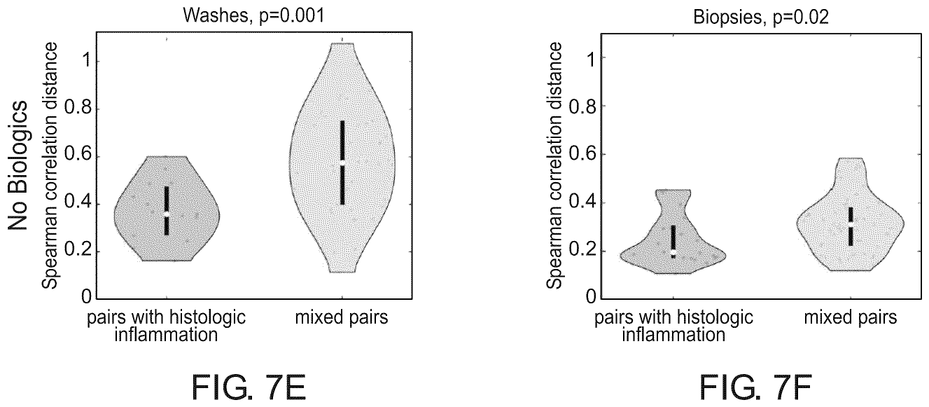

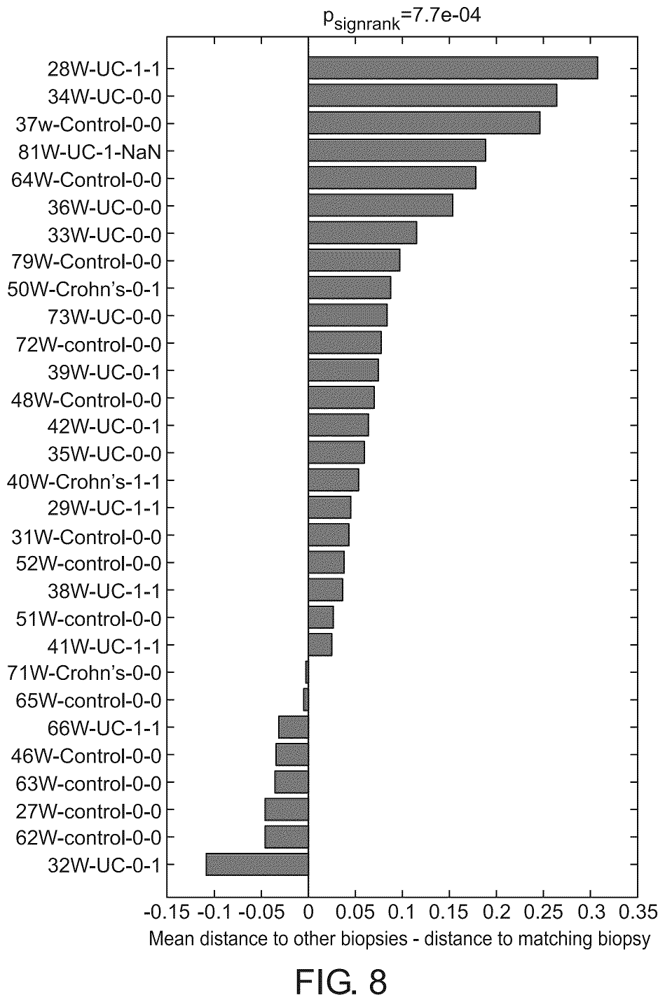

When assessing concordance of fecal washes and biopsies with endoscopic, rather than histological inflammation, similarly, fecal washes, rather than biopsies, were associated with endoscopic remission (p=0.004 versus p=0.6 respectively, FIG. 7A). Furthermore, statistically higher concordance of fecal wash transcriptomics with histological inflammation status was observed, compared to biopsy transcriptomics when stratifying according to patients' age or biological therapy (FIGS. 7B-C). The expression signatures of fecal washes were generally more similar to their matching biopsies than to other biopsies (FIG. 8)

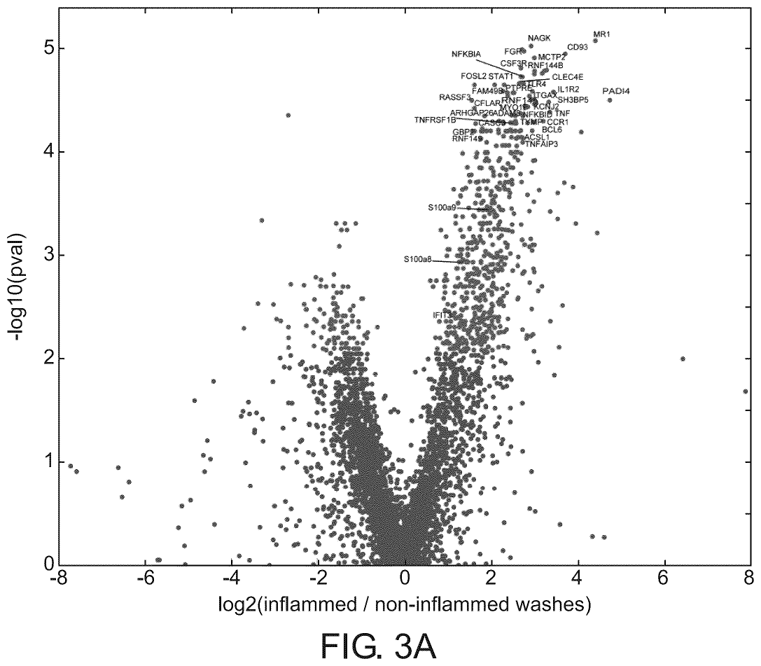

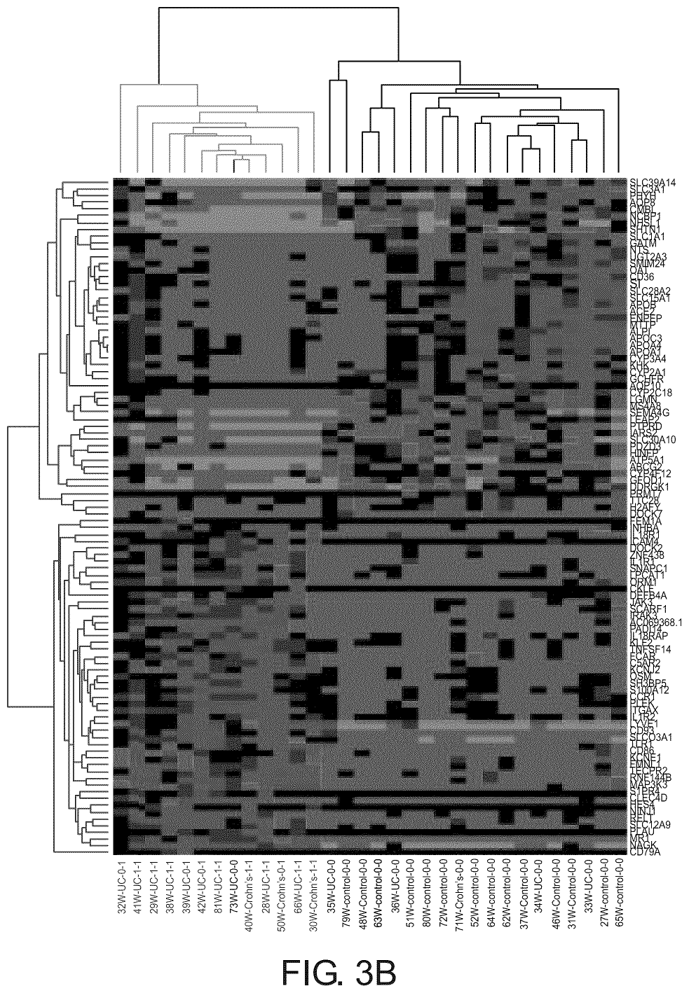

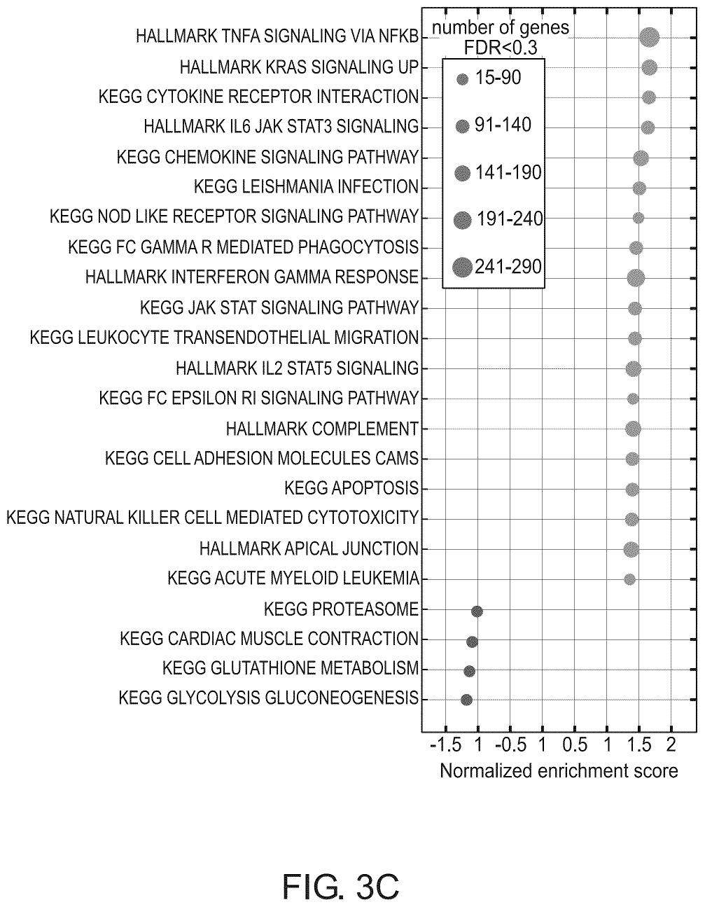

Gene expression patterns are significantly different between fecal washes of patients with and without histological inflammation: 1168 genes out of 3999 highly expressed genes were differentially expressed in fecal washes from patients with and without histological inflammation (FIGS. 3A, B normalized expression above 5*10⁻⁵ q-value<0.1, Wilcoxon rank sum tests with Benjamini-Hochberg false discovery rate correction). Genes that were upregulated in inflamed sample washes included S100A8 and S100A9, encoding the subunits of the calprotectin protein, as well as other immune-related genes such as NFKBIA, TNF, TNFRSF1B, CCR1, STAT1 and IFIT3. Using Gene Set Enrichment Analysis (GSEA)³² it was found that washes from inflamed patients were enriched in genes associated with TNFA signaling. IL6 signaling, chemokine signaling pathway and the JAK STAT pathway, and depleted in epithelial pathways such as glycolysis and glutathione metabolism (FIG. 3C).

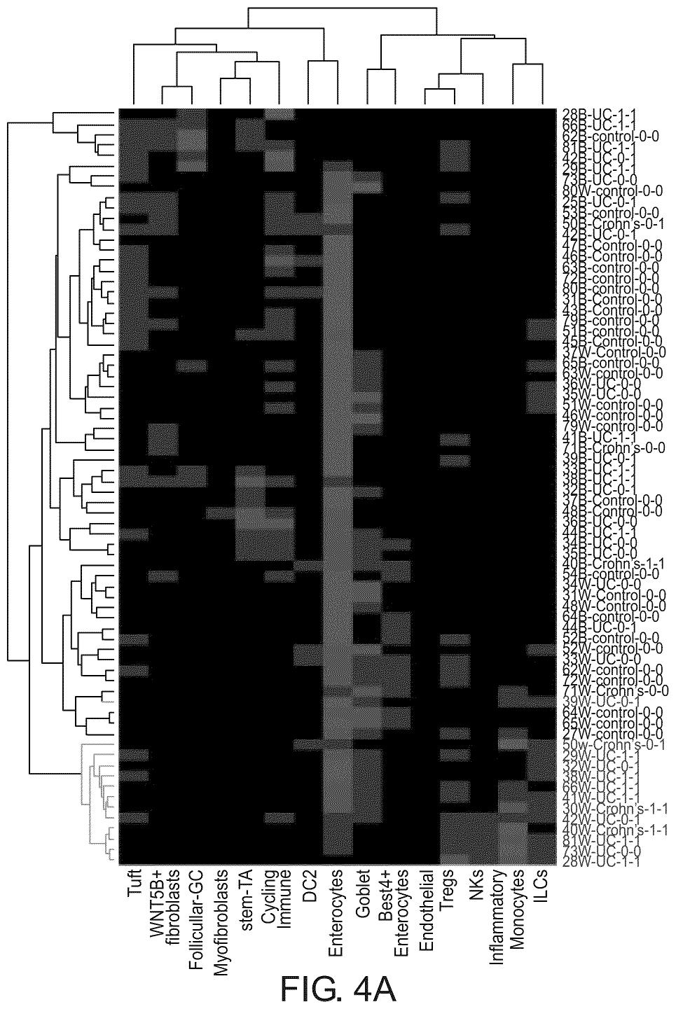

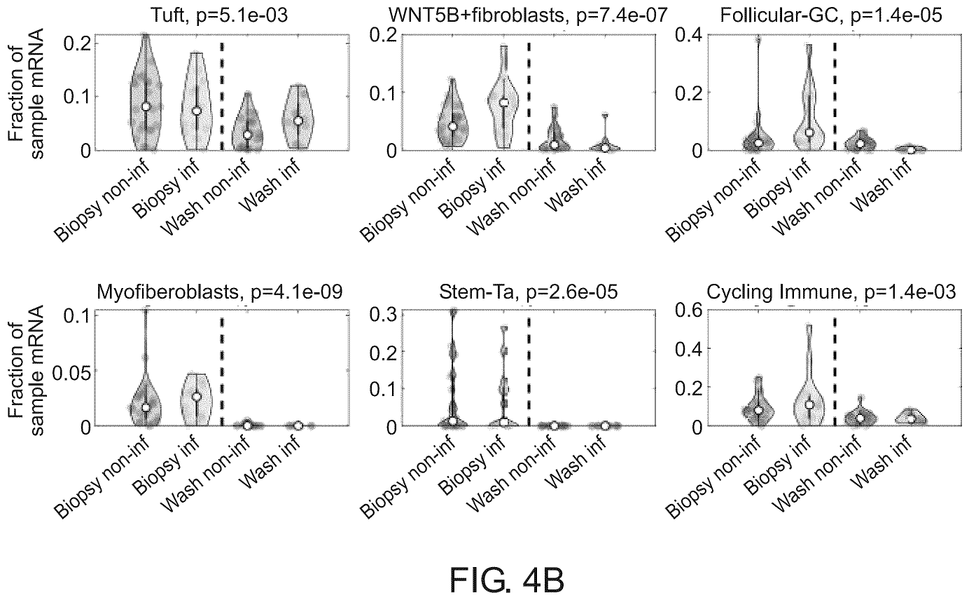

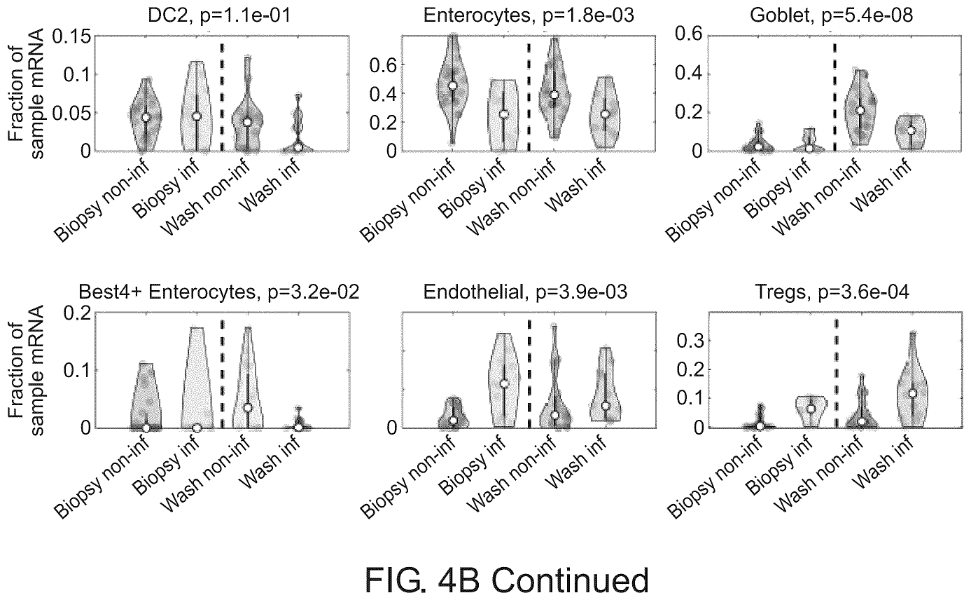

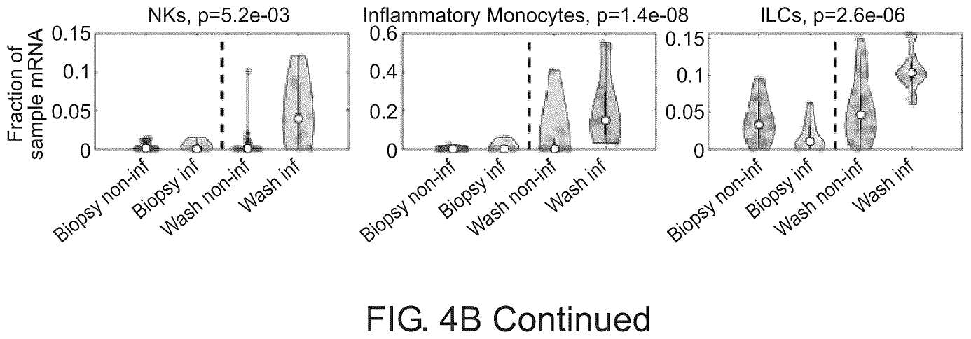

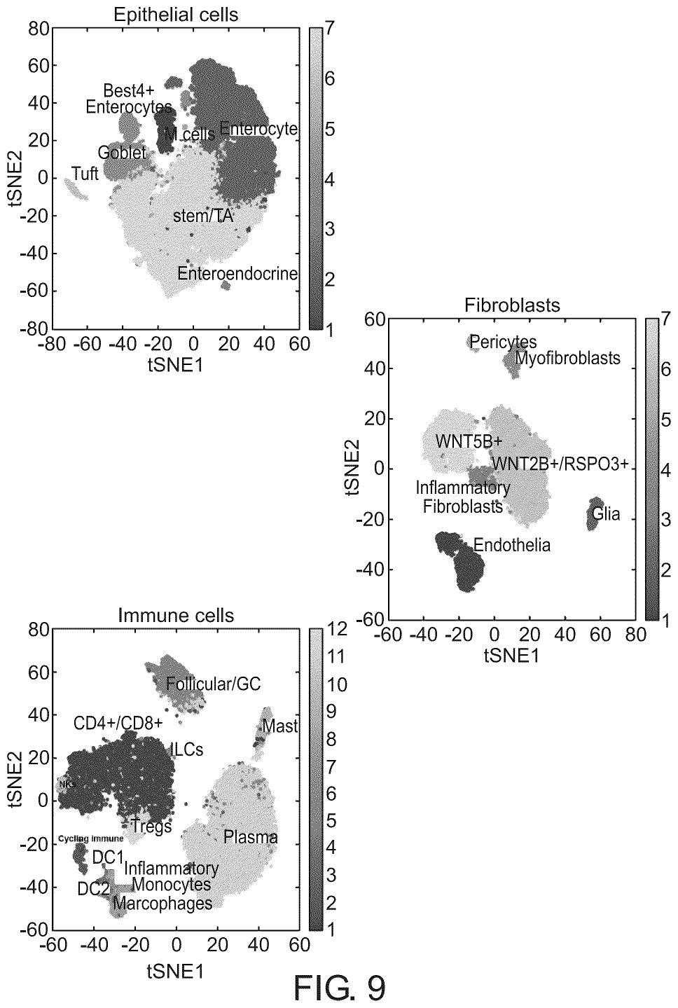

Inflamed fecal washes exhibit distinct cellular composition: cell compositions among inflamed versus non inflamed fecal washes and biopsies were inferred using CIBERSORTx²⁸ (Methods/Bioinformatic and computational analysis), using gene expression signatures of human colonic cell types that were parsed based on a recent single cell RNAseq study²⁹ (Methods, FIG. 9). An elevated representation of distinct immune cell subtypes were found in the washes of patients with histological inflammation (FIGS. 4A-B). Cell types that were elevated in fecal washes from patients with histological inflammation included regulatory T cells (p=2.1*104), natural killer (NK) cells (p=5.5*10⁻³), inflammatory monocytes (7.5*10⁻⁹) and innate lymphoid cells (ILCs, p=1.4*10⁻⁶). The increased differential representation of these immune subsets was higher in fecal washes when compared to biopsies (FIG. 4B). Non-inflamed washes had a significantly higher representation of enterocytes (p=2.7*10⁻³), myofibroblasts (2.1*10⁻⁹) and goblet cells (2.8*10⁻⁸) compared to inflamed washes.

More genes have expression levels that are highly predictive of histological inflammation in fecal washes compared to biopsies: The present inventors next sought to assess whether expression levels of individual genes can classify samples as belonging to patients with or without histological inflammation. The present inventors performed Receiver Operating Characteristic (ROC) curve analysis for all genes in the biopsies and fecal washes and examined the area under the curve (AUC). NFKBIA is demonstrated as an example (FIG. 5A). It was found that in the washes, the expression levels of multiple individual genes were significantly more predictive of histological inflammation compared to the biopsies. This was evident by the significantly higher AUC of the 5% genes with highest AUC levels in both groups (p=1.85*10⁻⁷², FIG. 5B). Fccal washes included 150 genes with AUC>0.9, whereas biopsies had only 10 such genes (FIG. 5C-E). Pathway analysis demonstrated that the 5% genes with the highest AUC in fecal washes were enriched for TNFα signaling via NF-KB, and inflammatory response, interferon α and γ signaling pathways, and IL-6/JAK/STAT signaling.

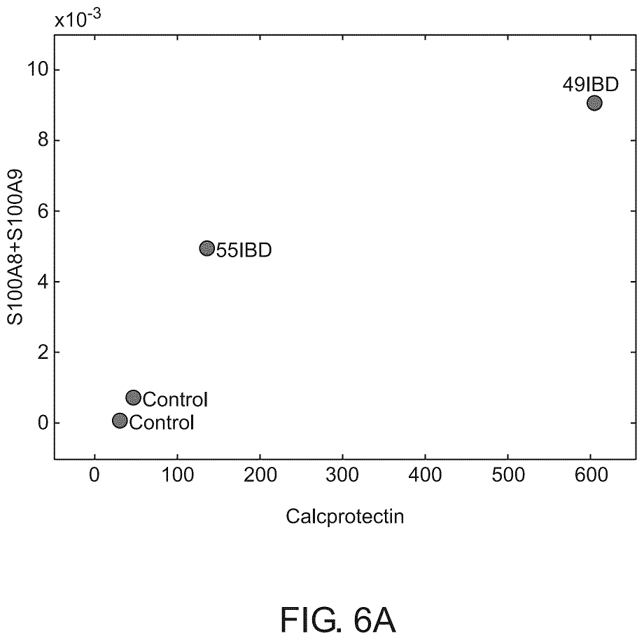

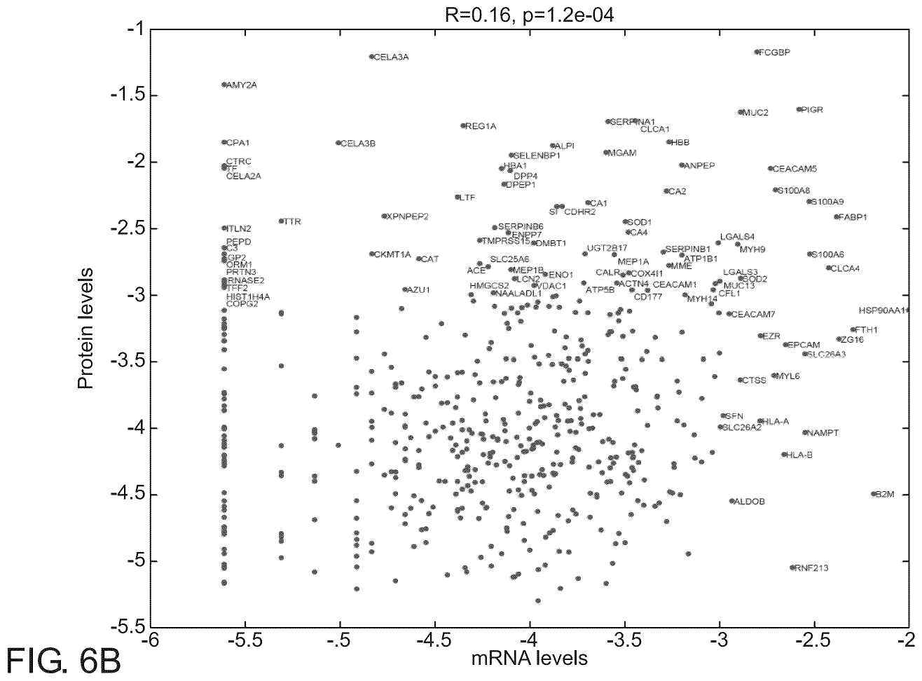

Fecal wash transcriptomics carries distinct information from fecal proteomics: To assess the information contained by the fecal wash transcriptomics measurements in relation to fecal proteomics, Mass Spectrometry Proteomics of 10 fecal samples (6 fecal washes and 4 stool samples) was performed. The six fecal washes had matching fecal wash transcriptomics analyses. Fecal calprotectin levels were measured in the 4 stool samples. Protein expression of S100A8 and S100A9 were correlated with stool calprotectin levels (FIG. 6A). Notably, protein and mRNA levels were only weakly correlated (R=0.16, p=1.2*10⁻⁴). Genes with discordant mRNA and protein levels included pancreatic proteins, such as the amylase protein AMY2A, and the elastase proteins CELA2A, CELA3A and CELA3B (FIG. 6B). These proteins are produced by pancreatic acinar cells and settle on the luminal side of the intestinal epithelium, explaining the lack of mRNAs. Other discordances may represent differential stability of distinct proteins and mRNA species. The fecal host transcriptomics therefore provides information that is distinct from fecal proteomics.

### Fecal Wash Transcriptomics Carries Information Regarding Histological Inflammation in the Ileum

In 10/11 patients with Crohn's disease in the ileum/right (proximal) colon left sided colonic washes demonstrated an inflammatory signature, similar to patients with left sided inflammation.

### EXAMPLE 2

### Materials and Methods

Sample collection and storage: Participants were given either a 15 ml tube with 5-10 ml of RNAlater, or a shaking 15 ml tube with 2 ml RLT (cell lysis buffer supplied in Qiagen RNeasy kits based on guanidinium thiocyonate) supplemented with DTT in a final concentration of 0.04 M. Collection tubes without the sample were kept at RT. Participants transferred 2 spoonfuls from a fresh stool sample, which has not passed into the toilet, into the collection tube. Sample size was 0.5 mm³*2 samples. The tube was shaken manually up and down for 60 seconds and were stored in a vertical position for 24-48 hours until delivery.

Collection tubes containing the samples were kept at 4° C. for at least 24 hours and not more than 48 hours, and were then frozen at −80° C. for at least 2 days. Content from shaking tube was transferred into two 2 ml Eppendorf tube prior to freezing (˜1 ml per tube).

RNA extraction: Prior to RNA extraction, samples were thawed on ice. Samples stored in RNAlater were transferred into a 2 ml Eppendorf, with as little residues of RNAlater as possible. A volume of 1 ml RLT+0.04M DTT was added to the sample. Samples frozen in RLT were thawed and additional RLT-DTT was added according to consistency.

All 2 ml Eppendorf tube containing sample and RLT+DTT were vigorously vortexed. 0.55 mm diameter RNase free zirconium-oxide homogenization (Next Advance) were added in a mass comparable to that of the fecal sample, and samples were homogenized in a Bullet Blender (Next Advance) using speed 8 setting for 3 mins. After the homogenization step, samples were centrifuged (200-500 rcf. 1-10 min) and 700 μl from the supernatant were transferred into a new Eppendorf tube. An equal volume of 100% EtOH is added to the sample and tube was vortexed. The content was then loaded on an RNeasy spin column placed in a 2 ml collection tube. RNA extraction steps were according to Qiagen's protocol for RNeasy micro kit with DNase I digestion step. Samples were eluted in 30 μl nuclease free water.

Samples stored in RNAlater were also transferred in the same manner into cold 700 μl TRI-reagent for RNA isolation (Sigma), and following the Bullet Blender homogenization steps described above, supernatant was transferred into a new tube. 100% EtOH was added an equal volume, and after vortexing, samples were loaded into Direct-zol RNA microprep column (Zymo research). RNA extraction was performed as detailed in kit protocol, with a DNase I incubation step. Samples were eluted in 30 μl nuclease free water.

### Negative Selection by Depletion on Non-Host RNA+DNA

RNA extracted from stool samples is comprised of transcripts of multiple species, including host (human) and commensal microorganisms such as microbiome, mycobiome and other parasite populations. As these populations outnumber the cells shed from the intestinal tract, only a minority of RNA content is originated in human. In order to enrich the readout from human RNA in an unbiased fashion, RNA molecules highly expressed by bacteria were depleted.

The depletion was carried out in three steps: first. DNA oligos designed and synthesized to be reverse-complement to bacterial transcripts with high expression level (such as bacterial rRNA—5S, 16S, 23S) were hybridized to the RNA. Next, RNase H enzyme digests and RNA-DNA specific hybrids, which leads to the selective digestion of only RNA molecules targeted by the DNA probes. Lastly, DNase I enzyme endonucleases the left over DNA probes and other DNA residues left in the sample after RNA extraction followed by RNA cleanup.

To deplete bacterial rRNA from our samples, the NEBNext rRNA Depletion Kit (Bacteria) kit and protocol was followed. After RNA purification step, bacterial rRNA depleted RNA is eluted in 8.4 μl nuclease free water and immediately proceeded to mcSCRBseq protocol for library preparation.

### Results

Stool samples represent particularly challenging starting biological material, due to their texture, potential long residence time of shed intestinal cells and elevated microbial content. Here, the present inventors have optimized protocol for the enrichment and successful sequencing of host mRNA. The key steps involved are sample acquirement and negative genomic selection of abundant microbial transcripts. This increases the fraction of human exonic reads in the sequenced samples (FIG. 11A). When applying this method on stool from both control donors and IBD patients with active inflammation, stool samples are clustered similarly to fecal wash samples, based on their presence of inflammation (FIG. 1B-C). Profound enrichment of the inflamed host transcriptomic signature observed in fecal washes (FIG. 11D-E) is also observed in stool samples following bacterial rRNA depletion demonstrating the ability of stool host transcriptomics to provide valuable information on IBD disease state.

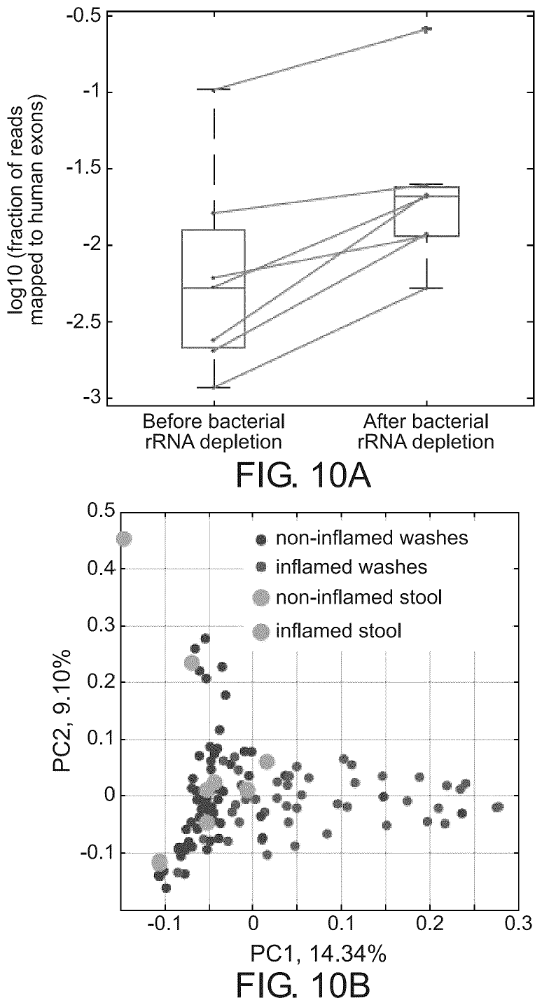

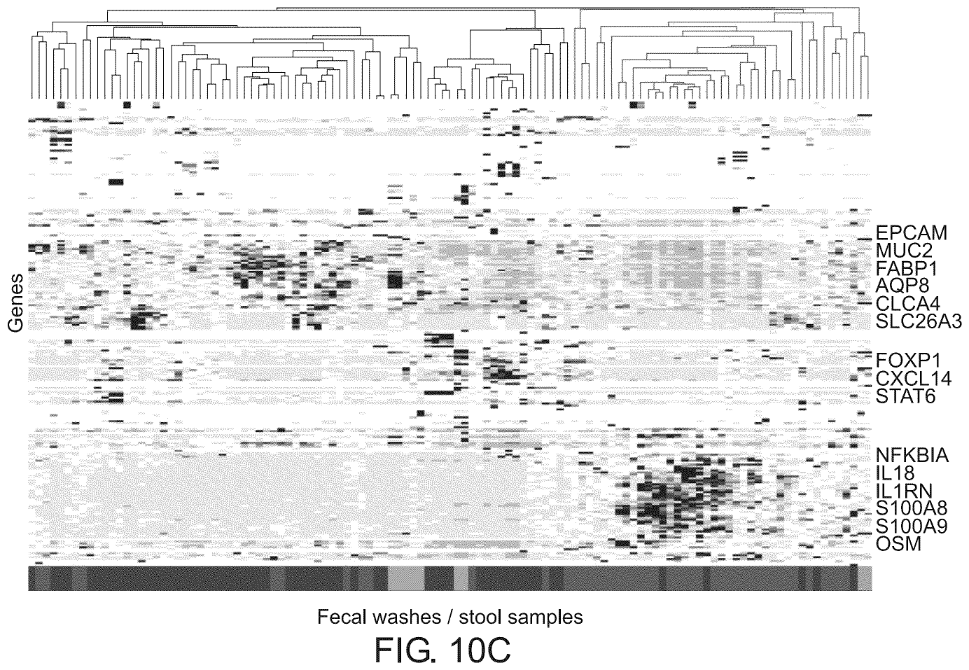

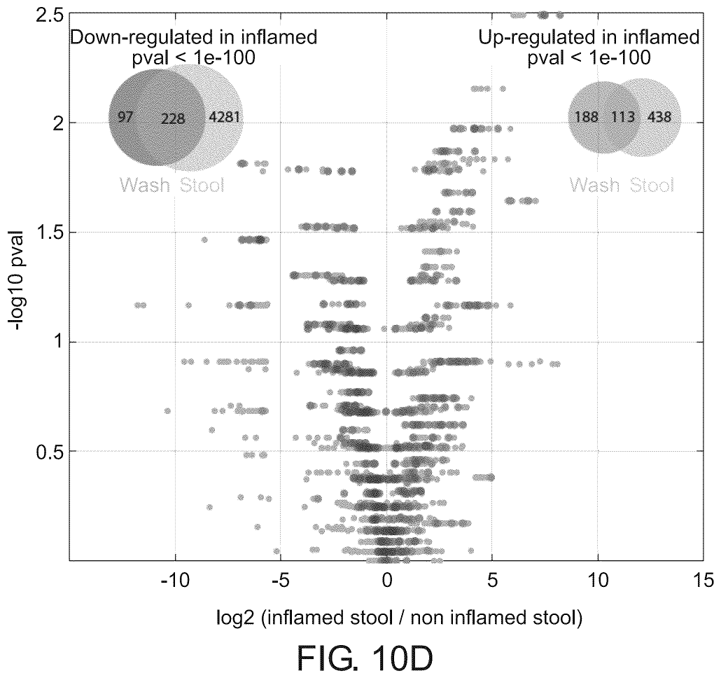

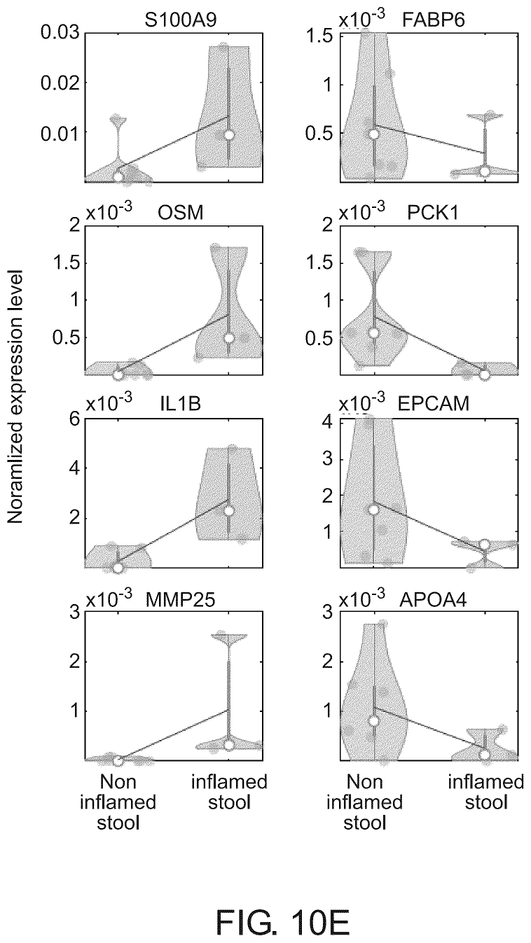

FIGS. 10A-E illustrate that bacterial rRNA depleted stool transcriptomics are informative is assessing intestinal inflammation.

Tables 5 and 6 provides a list of genes that were upregulated in the stool sample of subjects with Crohn's disease as compared to control (healthy subjects). Table 5 lists genes that were upregulated in stool samples and not in fecal washes in subjects with Crohn's disease as compared to control (healthy subjects) and Table 6 lists genes that were upregulated in both stool samples and in fecal washes in subjects with Crohn's disease as compared to control (healthy subjects).

log2(fold change) and Kruskal-Wallis p-values are given for stool samples (Table 5) and AUC score, together with log2(fold change) and FDR are given for fecal wash analysis (Table 6).

TABLE 5

| gene_name | stool_fc | stool_kw |
|---|---|---|
| AC008575.1 | 4.012444 | 0.181148 |
| AC012309.1 | 3.890391 | 0.1234 |
| ACTG2 | 3.34422 | 0.052705 |
| ADAM10 | 2.838591 | 0.016703 |
| ADAMTS7 | 4.581459 | 0.416075 |
| ADH1B | 4.351722 | 0.068342 |
| ADIPOR1 | 3.080441 | 0.045625 |
| AGPAT4 | 4.763037 | 0.007015 |
| AL159163.1 | 3.590142 | 0.299127 |
| ALB | 2.917742 | 0.052705 |
| ALOX5AP | 4.084485 | 0.122995 |
| ALPL | 3.814532 | 0.122995 |
| ANKRD37 | 3.192058 | 0.273913 |
| ANO9 | 3.795823 | 0.674022 |
| ARF6 | 1.349476 | 0.138477 |
| ARHGAP25 | 5.839025 | 0.02278 |
| ARL6IP6 | 4.406083 | 0.416075 |
| ASAP1 | 3.752227 | 0.020902 |
| ATAD2 | 3.587143 | 0.029065 |
| ATP2A2 | 2.749556 | 0.016375 |
| ATXN1 | 3.076367 | 0.39479 |
| B4GALT7 | 5.730217 | 0.02278 |
| BCL2A1 | 1.732472 | 0.086416 |
| BHMT | 4.047272 | 0.020902 |
| BID | 3.595198 | 0.674022 |
| C6 | 4.125298 | 0.122995 |
| CABIN1 | 5.730217 | 0.02278 |
| CALCR | 7.256459 | 0.122995 |
| CCDC30 | 4.433839 | 0.1234 |
| CCDC80 | 5.904456 | 0.02278 |
| CCM2L | 6.730926 | 0.02278 |
| CCNL2 | 3.332577 | 0.181148 |
| CD69 | 3.477145 | 0.010683 |
| CDC5L | 3.427251 | 0.1234 |
| CDH4 | 4.511769 | 0.416075 |
| CEBPB | 2.652899 | 0.346892 |
| CEP63 | 4.077074 | 0.020902 |
| CFD | 4.944315 | 0.416075 |
| CHD8 | 4.882972 | 0.068342 |
| CHORDC1 | 3.16004 | 0.010683 |
| CHRNA9 | 4.220119 | 0.416075 |
| CHSY1 | 5.764521 | 0.12663 |
| CLEC2B | 7.462035 | 0.003235 |
| CMAS | 3.882494 | 0.015405 |
| COTL1 | 3.608131 | 0.239678 |
| COX17 | 4.023623 | 0.068342 |
| CP | 4.696657 | 0.068342 |
| CPQ | 6.147138 | 0.003235 |
| CPS1 | 6.001291 | 0.003235 |
| CSF2RA | 5.839025 | 0.02278 |
| CXCL1 | 3.415884 | 0.1234 |
| CXCR1 | 6.694394 | 0.02278 |
| CXCR2 | 3.381869 | 0.199183 |
| CYBA | 3.454691 | 0.025483 |
| CYTH4 | 4.770372 | 0.068342 |
| DAB1 | 3.961348 | 0.068342 |
| DDRGK1 | 3.605296 | 0.1234 |
| DNAJC19 | 5.111876 | 0.014713 |
| DNAJC25- | 4.454861 | 0.010683 |
| GNG10 | | |
| DNAJC7 | 2.877675 | 0.030368 |
| EIF3M | 3.425623 | 0.010683 |
| EIF5B | 5.50752 | 0.007015 |
| ELOVL5 | 6.279584 | 0.003235 |
| EMC9 | 3.323896 | 0.03877 |
| EPHX2 | 3.239673 | 0.304371 |
| ERLEC1 | 3.319693 | 0.39479 |
| EXOC2 | 6.001291 | 0.003235 |
| FAAH | 4.245561 | 0.014713 |
| FAM173B | 7.02232 | 0.02278 |
| FAM3C | 5.740035 | 0.014713 |
| FAM81B | 5.839025 | 0.02278 |
| FCGR3A | 7.472449 | 0.003235 |
| FCGR3B | 3.904839 | 0.025483 |
| FGG | 3.278153 | 0.025483 |
| FLVCR1 | 2.950547 | 0.016703 |
| FPR2 | 3.180417 | 0.010683 |
| FZD3 | 5.860595 | 0.010683 |
| GALNT7 | 3.786802 | 0.199183 |
| GBP5 | 8.200239 | 0.003235 |
| GNB4 | 5.934898 | 0.02278 |
| GPM6A | 1.350438 | 0.138477 |
| GPRC5C | 4.082265 | 0.029868 |
| GREM1 | 1.431908 | 0.305059 |
| GRIP2 | 2.872483 | 0.028381 |
| HBA1 | 4.751018 | 0.416075 |
| HBD | 4.871985 | 0.068342 |
| HCAR2 | 2.993923 | 0.239678 |
| HCST | 5.833339 | 0.02278 |
| HDHD3 | 2.047872 | 0.087375 |
| HEPN1 | 4.572701 | 0.016703 |
| HP1BP3 | 3.209212 | 0.015405 |
| IBTK | 3.545361 | 0.304371 |
| IFITM3 | 2.531645 | 0.016375 |
| IPO7 | 4.751018 | 0.416075 |
| IQSEC1 | 4.553071 | 0.068342 |
| IREB2 | 4.220119 | 0.416075 |
| ITGA1 | 3.528575 | 0.674022 |
| ITIH2 | 3.811671 | 0.068342 |
| KIAA1324 | 3.775384 | 0.068342 |
| KLHL36 | 3.740056 | 0.068342 |
| LAPTM5 | 3.210879 | 0.239678 |
| LILRB3 | 7.308618 | 0.003235 |
| LPP | 2.886075 | 0.030368 |
| LSP1 | 4.258289 | 0.010683 |
| LTA4H | 6.580425 | 0.02278 |
| MED31 | 6.001291 | 0.003235 |
| METTL23 | 4.123615 | 0.007015 |
| MMP25 | 5.261158 | 0.010683 |
| MNDA | 3.758398 | 0.1234 |
| MRPL18 | 5.172648 | 0.068342 |
| MTMR4 | 5.839025 | 0.02278 |
| MYH11 | 4.808434 | 0.068342 |
| NABP1 | 2.221682 | 0.052705 |
| NCF2 | 3.124664 | 0.181148 |
| NMT1 | 4.165758 | 0.068342 |
| NOD1 | 4.831902 | 0.010683 |
| NRN1 | 3.89054 | 0.015405 |
| OAT | 3.230242 | 0.239678 |
| OBSCN | 4.256406 | 0.416075 |
| OSM | 4.150752 | 0.010683 |
| OTX1 | 6.28376 | 0.12663 |
| P3H4 | 3.77058 | 0.674022 |
| PABPC3 | 4.2293 | 0.007015 |
| PAM | 7.067748 | 0.02278 |
| PCP4 | 5.839025 | 0.02278 |
| PDLIM7 | 5.856346 | 0.068342 |
| PDZD3 | 3.93765 | 0.014713 |
| PHF7 | 6.147138 | 0.003235 |
| PIF1 | 3.784729 | 0.029065 |
| PISD | 5.934898 | 0.02278 |
| PLCB1 | 4.917286 | 0.122995 |
| PLK3 | 4.035947 | 0.020902 |
| PLXNC1 | 3.365697 | 0.1234 |
| PRKRA | 4.286296 | 0.068342 |
| PROM1 | 4.406083 | 0.416075 |
| PRR29 | 4.259367 | 0.014713 |
| PSMD6 | 3.645424 | 0.068342 |
| PSME4 | 4.468549 | 0.007015 |
| PTPDC1 | 3.666576 | 0.068342 |
| PYGL | 3.582696 | 0.1234 |
| RBM26 | 3.645109 | 0.010683 |
| RDX | 4.574894 | 0.014713 |
| RGS19 | 6.470194 | 0.003235 |
| RHOH | 3.696731 | 0.068342 |
| RPS23 | 2.740785 | 0.015405 |
| S100A12 | 4.168437 | 0.068342 |
| S100A4 | 3.398171 | 0.016375 |
| S100A7 | 3.867678 | 0.674022 |
| SEPT1 | 3.852541 | 0.068342 |
| SLC19A3 | 6.862537 | 0.12663 |
| SLK | 3.158031 | 0.016375 |
| SMC6 | 3.811671 | 0.068342 |
| SNRPE | 6.354815 | 0.003235 |
| SNX27 | 3.566325 | 0.010683 |
| SPRR2E | 3.049186 | 0.674022 |
| SRSF10 | 4.559213 | 0.010683 |
| STAG1 | 3.253366 | 0.1234 |
| TBC1D10B | 4.056729 | 0.025483 |
| TEPP | 4.220119 | 0.416075 |
| TIMM10B | 3.811671 | 0.068342 |
| TMEM147 | 4.871985 | 0.068342 |
| TMEM33 | 3.649542 | 0.199183 |
| TMPRSS5 | 7.856324 | 0.12663 |
| TNFRSF14 | 3.997344 | 0.897842 |
| TSPAN5 | 3.625116 | 0.014713 |
| TTN | 2.955634 | 0.086416 |
| TXNL4B | 6.404727 | 0.02278 |
| TYW1B | 6.536324 | 0.02278 |
| UIMC1 | 5.839025 | 0.02278 |
| UQCC2 | 4.220119 | 0.416075 |
| UQCRHL | 3.549122 | 0.239678 |
| USB1 | 3.913744 | 0.1234 |
| USP1 | 3.398703 | 0.020902 |
| USP28 | 8.077827 | 0.12663 |
| WARS | 4.364866 | 0.068342 |
| XPO4 | 4.165758 | 0.068342 |
| XPO6 | 3.813945 | 0.1234 |
| ZFP41 | 6.28376 | 0.12663 |
| ZNF235 | 4.306729 | 0.068342 |
| ZSWIM8 | 3.761562 | 0.068342 |

TABLE 6

| | | | | | AUC |
|---|---|---|---|---|---|
| Gene name | Wash fc | Wash q | Stool fc | Stool kw | wash |
| AC007192.1 | 2.601391 | 2.66E−07 | 3.140936 | 0.025483 | 0.88 |
| APBB1IP | 2.066958 | 1.09E−05 | 3.932571 | 0.068342 | 0.9 |
| ARHGAP30 | 1.966468 | 0.000256 | 5.833339 | 0.02278 | 0.85 |
| CSF3R | 2.871634 | 1.09E−07 | 3.129623 | 0.1234 | 0.98 |
| CXCL8 | 2.725216 | 2.66E−07 | 2.157218 | 0.052705 | 0.9 |
| EVI2B | 2.128138 | 3.10E−07 | 4.2872 | 0.068342 | 0.92 |
| FYB1 | 2.528831 | 2.26E−07 | 2.926725 | 0.273913 | 0.94 |
| G0S2 | 2.390331 | 2.66E−07 | 3.277559 | 0.052705 | 0.92 |
| IFITM2 | 1.954758 | 4.47E−06 | 2.195922 | 0.030368 | 0.86 |
| IL1B | 2.721195 | 2.66E−07 | 3.396133 | 0.013543 | 0.91 |
| JAML | 1.991764 | 6.22E−05 | 4.239473 | 0.122995 | 0.88 |
| MYO1F | 2.6196 | 2.66E−07 | 3.892352 | 0.068342 | 0.95 |
| PLEK | 2.987803 | 1.09E−07 | 2.361776 | 0.04985 | 0.92 |
| PROK2 | 3.009313 | 3.48E−07 | 4.172997 | 0.013543 | 0.94 |
| PTPRC | 2.135346 | 4.08E−07 | 4.133807 | 0.010683 | 0.91 |
| S100A8 | 1.982975 | 1.64E−05 | 2.042953 | 0.087375 | 0.86 |
| S100A9 | 2.169289 | 2.27E−06 | 2.275536 | 0.052705 | 0.9 |
| SOCS3 | 2.816118 | 1.90E−07 | 3.194971 | 0.028381 | 0.95 |
| SRGN | 2.205152 | 5.10E−07 | 2.640635 | 0.029868 | 0.9 |
| TMEM154 | 1.889821 | 1.40E−06 | 4.148742 | 0.010683 | 0.94 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1-25. (canceled)

26. A method of diagnosing an inflammatory bowel disease (IBD) of a subject comprising analyzing the RNA expression level of at least one human gene in a fecal RNA sample of the subject, wherein the method further comprises depleting said sample of microbial RNA prior to the analyzing, and wherein when the expression level of a human gene set forth in Table 1, 5 or 6 is statistically significantly altered over the level of said gene in a fecal RNA sample of a control subject, it is indicative of the inflammatory bowel disease.

27. The method of claim 26, wherein the depletion comprises negative genomic selection.

28. The method of claim 26, wherein said depletion comprises depletion of at least one, at least two, at least three, at least four or at least five of the following bacteria: Eubacterium rectale, Faecalibacterium prausnitzii, Bifidobacterium adolescentis, Ruminococcus sp 5 1 39BFAA, Bifidobacterium longum, Subdoligranulum, Ruminococcus gnavus, Escherichia coli, Ruminococcus torques, Akkermansia muciniphila, Ruminococcus bromii, Dialister invisus, Collinsella aerofaciens, Bacteroides uniformis, Bacteroides vulgatus, Eubacterium hallii, Dorea longicatena, Prevotella copri, Alistipes putredinis and Bifidobacterium bifidum.

29. The method of claim 26, wherein said sample is a solid fecal sample.

30. The method of claim 26, wherein at least five human RNA determinants of Table 1, 5 or 6 are analyzed.

31. The method of claim 26, wherein at least ten human RNA determinants of Table 1, 5 or 6 are analyzed.

32. The method of claim 26, wherein said IBD comprises ulcerative colitis or Crohn's colitis.

33. The method of claim 26, wherein said diagnosing the IBD comprises determining the severity of the IBD.

34. The method of claim 26, further comprising administering to the subject, if confirmed as having IBD, a therapeutically effective amount of an agent useful for treating the IBD.

35. A method of diagnosing a disease of the gastrointestinal tract of a subject comprising analyzing the expression level of at least one human gene in a fecal wash of the subject, wherein said analyzing is effected at the RNA level, and wherein the expression level is indicative of the disease of the gastrointestinal tract.

36. The method of claim 35, wherein said at least one human gene is selected from Table 1, 5 or 6.

37. The method of claim 36, wherein at least five human RNA determinant of Table 1, 5 or 6 are analyzed, or wherein at least ten human RNA determinant of Table 1, 5 or 6 are analyzed.

38. The method of claim 35, wherein said fecal wash is of the sigmoid colon of the subject, or wherein said fecal wash is of the rectum of the subject.

39. The method of claim 35, wherein the disease is an inflammatory bowel disease (IBD).

40. The method of claim 35, wherein the disease is a colon cancer or irritable bowel syndrome.

41. The method of claim 35, wherein said diagnosing the IBD comprises determining the severity of the IBD.

42. The method of claim 35, further comprising administering to the subject, if confirmed as having the disease, a therapeutically effective amount of an agent useful for treating said disease.

43. A method of analyzing the RNA expression level of at least one human gene in a solid fecal RNA sample of a subject, comprising depleting said fecal RNA sample of microbial RNA prior to the analyzing by negative genomic selection of bacterial and/or fungal RNA.

44. The method of claim 43, wherein the sample has been obtained from a subject suspected of having a disease of the gastrointestinal tract selected from the group consisting of inflammatory bowel disease (IBD), colon cancer and irritable bowel syndrome.

45. The method of claim 43, wherein said depletion comprises depletion of at least one, at least two, at least three, at least four or at least five of the following bacteria: Eubacterium rectale, Faecalibacterium prausnitzii, Bifidobacterium adolescentis, Ruminococcus sp 5 1 39BFAA, Bifidobacterium longum, Subdoligranulum, Ruminococcus gnavus, Escherichia coli, Ruminococcus torques, Akkermansia muciniphila, Ruminococcus bromii, Dialister invisus, Collinsella aerofaciens, Bacteroides uniformis, Bacteroides vulgatus, Eubacterium hallii, Dorea longicatena, Prevotella copri, Alistipes putredinis and Bifidobacterium bifidum.


SEQUENCE LISTING (24 sequences)

SEQ ID NO: 1 - RNA, 1521 nt, Eubacterium rectale
  source 1..1521: mol_type = rRNA
agagtttgatcctggctcaggatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggctcgataactggggtgaagtcgtaacaaggtagccgtatcggaaggtgcggctggatcacct

SEQ ID NO: 2 - RNA, 1462 nt, Faecalibacterium prausnitzii
  source 1..1462: mol_type = rRNA
gatcctggctcaggcgaacgctggcggcgcgcctaacacatgcaagtcgaacgagcgagagagagcttgctttctcaagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaaggcaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtggccgatgtttactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacgcacatagaaatatgtgtttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtac

SEQ ID NO: 3 - RNA, 1530 nt, Bifidobacterium adolescentis
  source 1..1530: mol_type = rRNA
tttgtggagggttcgattctggctcaggatgaacgctggcggcgtgcttaacacatgcaagtcgaacgggatcggctggagcttgctccggccgtgagagtggcgaacgggtgagtaatgcgtgaccgacctgccccatacaccggaatagctcctggaaacgggtggtaatgccggatgctccagttggatgcatgtccttctgggaaagattctatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacctcgtgagggggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctttttggggggagccgtctaaggtgagactcgtgattgggactaagtcgtaacaaggtagccgtaccggaaggtgcggctggatcacctccttt

SEQ ID NO: 4 - RNA, 1503 nt, Ruminococcus albus
  source 1..1503: mol_type = rRNA
agagtttgatcctggctcaggacgaacgctggcggcacgcttaacacatgcaagtcgaacgagcgaaagagtgcttgcactctctagctagtggcggacgggtgagtaacacgtgagcaatctgcctttcggagagggataccaattggaaacgattgttaatacctcataacataacgaagccgcatgactttgttatcaaatgaatttcgccgaaagatgagctcgcgtctgattaggtagttggtgaggtaacggcccaccaagccgacgatcagtagccggactgagaggttgaacggccacattgggactgagacacggcccagactcctacgggaggcagcagtggggaatattgcacaatgggcgaaagcctgatgcagcgatgccgcgtgagggaagaaggttttaggattgtaaacctctgtctttggggacgataatgacggtacccaaggaggaagctccggctaactacgtgccagcagccgcggtaatacgtagggagcgagcgttgtccggaattactgggtgtaaagggagcgtaggcgggattgcaagtcaggtgtgaaatttaggggcttaacccctgaactgcacttgaaactgtagttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggctgaaactcaaaggaattgacgggggcccgcacaagcagtggagtatgtggtttaattcgaagcaacgcgaagaaccttaccaggtcttgacatcgtacgcatagcatagagatatgtgaaatcccttcggggacgtatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggctacacacgtactacaatggctgttaacagagggaagcaaaacagtgatgtggagcaaaaccctaaaagcagtcttagttcggattgtaggctgcaacccgcctacatgaagtcggaattgctagtaatcgcggatcagcatgccgcggtgaatacgttcccgggccttgtacacaccgcccgtcacgccatgggagtcggtaacacccgaagcctgtgttctaaccgcaaggaggaagcagtcgaaggtgggattgatgactggggtgaagtcgtaacaaggtagccgtatcggaaggtgcggctggatcacctcctt

SEQ ID NO: 5 - RNA, 1464 nt, Bifidobacterium longum
  source 1..1464: mol_type = rRNA
ctggctcaggatgaacgctggcggcgtgcttaacacatgcaagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgt

SEQ ID NO: 6 - RNA, 1428 nt, Subdoligranulum variabile
  source 1..1428: mol_type = rRNA
tgcaagtcgaacggagttatttcggttgaagttttcggatggatactggtttaacttagtggcgaacgggtgagtaacgcgtgagtaacctgccctggagtgggggacaacagttggaaacgactgctaataccgcataagcccacgatccggcatcggattgagggaaaaggatttattcgcttcaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgaatcttgacggtacctaacaagaaagcaccggctaactacgtgccagcagccgcggtaaaacgtagggtgcaagcgttgtccggaattactgggtgtaaagggagcgcaggcggaccggcaagttggaagtgaaatctatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagtgcagagatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagangcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccgggggggacccgaagtcggtaagtaagtctaaccgcaaggaggacgccgccgaaggtaaaactggtgattgggtg

SEQ ID NO: 7 - RNA, 1481 nt, Ruminococcus gnavus
  source 1..1481: mol_type = rRNA
tgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacgnctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcancccctatctttagtagccagcattttggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgtncccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaagg

SEQ ID NO: 8 - RNA, 1450 nt, Escherichia coli
  source 1..1450: mol_type = rRNA
agtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagcacaaagagggggaccttagggcctcttgccatcggatgtgcccagatgggattagctagtaggtggggtaacggctcacctaggcgacgatccctagctggtctgagaggatgaccagcaacactggaactgagacacggtccagactcctacgggaggcagcagtggggaatattgcacaatgggcgcaagcctgatgcagccatgcngcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccgganntaacgcgttaagtcgaccgcctggggagtacggccgcaaggttaaaactcaaatgaattgacgggggccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaacttcgggagggcg

SEQ ID NO: 9 - RNA, 1418 nt, Ruminococcus torques
  source 1..1418: mol_type = rRNA
ctcaggatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgtngaggctcgaaagcgtggggagcacacaggattagataccctggtagnccacnccgtaaacgatgactactaggtgtcgggtgncaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtcccgcaacgagcgcaacccctatctttagtagccagcggccaggccgggcactctagagagactgccggggataacccggaggaaggtggggatgacgtcaaatcatcatgccccttatgagcagggctacacacgtgctacaatggcgtaaacaaagggaagcgagaccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgt

SEQ ID NO: 10 - RNA, 1433 nt, Akkermansia muciniphila
  source 1..1433: mol_type = rRNA
aacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagtaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagctggtcgcacccgaagtatctgaagccaaccgcaaggaggcagggtcctaaggtgagactggtaactgggatg

SEQ ID NO: 11 - RNA, 1380 nt, Ruminococcus bromii
  source 1..1380: mol_type = rRNA
tcgaacggaantgttttgaaagatttcttcggaatgaatttgatttagtttagtggcggacgggtgagtaacgcgtgagtaacctgccttcaagagggggataacattctgaaaagaatgctaataccgcatgacatatcggaaccacatggttctgatatcaaagattttatcgcttgaagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggcaaccctgacgcaccaacgccgcgtgaaggatgaaggttttcggattgtaaacttcttttattaaggacgaaaaatgacggtacttaatgaataagctccggctaactacgtgccagcagccgcggtaatacgtagggagcaagcgttgtccggatttactgggtgtaaagggtgcgtaggcggctttgcaagtcagatgtgaaantctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggagggtctgaccccttccgtgccggagttaacacaataagtaatccacctggggagtacggmcnnaargttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttggkttaagtcccgcaacgagcgcaacccttgctattagttgntacgcaagagcactctaataggactgccgttgacaaaacggaggaaggtggggacgacgtcaaatcatcatgccccttatgacctgggctacacacgtactacaatggatgttaacagagggaagcaagacagtgatgtggagcaaacccctaaaaacattctcagttcagattgcaggctgcaacccgcctgcatgaagatggaattgctagtaatcgnggatcagaatgmcgcggtgaatacgttcccgggccttgtacacaccgcccgtcacaccatgggagccggtaatacccgaagtcagtagtccaacctc

SEQ ID NO: 12 - RNA, 1524 nt, Dialister invisus
  source 1..1524: mol_type = rRNA
gacgaacgctggcggcgtgcttaacacatgcaagtcgaacgaaaagacggaaagagcttgctcttttcagaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttaccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgaggaagagcaaacctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaaggcggagctgatgattggagtgaagtcgtaacaaggtagccgtatcggaaggtgc

SEQ ID NO: 13 - RNA, 1463 nt, Collinsella aerofaciens
  source 1..1463: mol_type = rRNA
agagtttgatcctggctcaggatgaacgctggcggcgcgcctaacacatgcaagtcgaacggcacccctctccggagggaagcgagtggcgaacggctgagtaacacgtggagaacctgccccctcccccgggatagccgcccgaaaggacgggtaataccggataccccggggtgccgcatggcgccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtagggagggtggaacaccgggtgtagcggtggaatgcgcagatatcgggtggaacaccgatggcgaaggcggccctctgggccaagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaagntaaaactcaaaggaattgacgggggcccgcacaagcagcggagcatgtggcttaattcgaagcaacgcgaagaaccttaccagggcttgacatatgggtgaagcgggggagacccgtggccgagaggagcccatacaggtggtgcatggctgtcgtcagctcgtgtcgtgagatgttgggttaagtcccgcaacgagcgcaacccccgccgcgtgttgccatcgggtgatgccgggaacccacgcgggaccgccgccgtcaaggggaggagggccgggacgacgtcaagtcatcatgccccttatgccctgggctgcacacgtgctacaatggccggtacagagggatgccaccccgcgaggggagcggatcccggaaagccggccccagttcggattgggggctgcaacccgcccccatgaagtcggagttgctagtaaacgcggatcagcatgccgcggtgaatgcgttcccgggccttgtacacaccggccgtcacaccacccgagtcgtctgcacccgaagtcgccggcccaaccgagaggggggaagcgccgaaggtgtggaaggttaagggggttaagtcgtaacaaaggtaacc

SEQ ID NO: 14 - RNA, 1425 nt, Bacteroides uniformis
  source 1..1425: mol_type = rRNA
gtttgatcatggctcaggatgaacgctagtacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaa

SEQ ID NO: 15 - RNA, 1283 nt, Bacteroides vulgatus
  source 1..1283: mol_type = rRNA
ttactagcgaatccagcttcgtggagtcgggttgcagactccagtccgaactgagagaggttttggggattggcatccactcgcgtggtagcggccctctgtaccccccattgtaacacgtgtgtagccccggacgtaagggccgtgctgatttgacgtcatccccaccttcctcacatcttacgatggcagtcttgtcagagtcctcagcgtgacctgttagtaactgacaacaagggttgcgctcgttatggcacttaagccgacacctcacggcacgagctgacgacaaccatgcagcaccttcacagatgccttgcggcttacggctttcaccgtaattcatctgcaatttaagcccgggtaaggttcctcgcgtatcatcgaattaaaccacatgttcctccgcttgtgcgggcccccgtcaattcctttgagtttcaccgttgccggcgtactccccaggtggaatacttaacgctttcgcttggccgcttgctgtatatcgcaaacagcgagtattcatcgtttaccgtgtggactaccagggtatctaatcctgtttgatacccacactttcgagcctcaatgtcagttgcagcttagcaggctgccttcgcaatcggagttcttcgtgatatctaagcatttcaccgctacaccacgaattccgcctgcctcaactgcactcaagatatccagtatcaactgcaattttacggttgagccgcaaactttcacaactgacttaaacatccatctacgctccctttaaacccaataaatccggataacgctcggatcctccgtattaccgcggctgctggcacggagttagccgatccttattcataaagtacatgcaaacgggtatccatacccgactttattcctttataaaagaagtttacaacccatagggcagtcatccttcacgctacttggctggttcaggctctcgcccattgaccaatattcctcactgctgcctcccgtaggagtttggaccgtgtctcagttccaatgtgggggaccttcctctcagaacccctatccatcgaagactaggtgggccgttaccccgcctactatctaatggaacgcatccccatcgtctaccggaatacctttaatcatgtgaacatgcggactcatgatgccatcttgtattaatcttcctttcagaaggctgtccaagagtagacggcaggttggatacgtgttactcacccgtgcgccggtcgccatcggccttagcaagctaagaccatgctgcccctcga

SEQ ID NO: 16 - RNA, 1479 nt, Eubacterium hallii
  source 1..1479: mol_type = rRNA
tttatttgagagtttgatcctggctcaggatgaacgctggcggcgtgcctaacacatgcaagtcgaacgaagcaccttaccwgattcttcggatgaaagwytggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggngctgcatttgaaactgcwyrgctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggctycggtgccgccgctaacgcagtaagtattccacctggggagtacgttcgcaagaatgaaactcaaaggaattgacggggacccgcacaagcggtggagcatgtggtttaattcgaagnaacgcgaagaaccttaccaggtcttgacatccttctgaccgcaccttaatcggtgctttccttcgggacagaagagacaggtggtgcatggttgtcgtcagctcgtgtcgtgagatgttgggttaagtcccncaacgagcgcnacccctatcttcagtagccagcaggtaaggctgggcactctggagagactgccagggataacctggaggaaggtggggacgacgtnnaatcatcatgccccttatgatctgggcgacacacgtgctacnatggcggtcacagagtgaggcgaaccygcganggggagcaanccacaaaaaggccgtcccagttcggactgtagtctgcaacccgactacacgaagctggaatcgctagtaatcgcgaatcagaatgtcgcggtgaatacgttcccnngtcttgtacacaccgnccgtcacaccatgggagtcggaaatgcccgaagccagtgacccaacctttatggagggagctgtcgaaggtggagccggtaactgggg

SEQ ID NO: 17 - RNA, 1316 nt, Dorea longicatena
  source 1..1316: mol_type = rRNA
taacgcgtgggtaacctgcctcatacagggggataacagttagaaatgactgctaataccgcataagaccacgtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagtttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcataacgcccgaagtcagtgacccaaccgtaagg

SEQ ID NO: 18 - RNA, 1494 nt, Prevotella copri
  source 1..1494: mol_type = rRNA
agagtttgatcctggctcaggatgaacgctagctacaggcttaacacatgcaagtcgaggggaaacgacatcgaaagcttgcttttgatgggcgtcgaccggcgcacgggtgagtaacgcgtatccaacctgcccaycacttggggataaccttgcgaaagtaagactaatacccaatgatatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatggrcgagagcctgaaccagccaagtagcgtgcaggatgacggccctatgggttgtaaactgcttttataagggaataaagtgagcctcgtgaggctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaacaggtcagcggccaagcgaaagcattaagcatcccacctggggagtacgccggcaacggtgaaactcaaaggaattgacgggggcccgcacaagcggaggaacatgtggtttaattcgatgatacgcgaggaaaccttacccgggcttgaattgcagaggaaggatttggagacaatgacgcccttcggggyctctgtgaaggtgctgcatggttgtcgtcagctcgtgccgtgaggtgtcggcttaagtgccataacgagcgcaacccctctccttagttgccatcaggtyaagctgggcactctggggacactgccaccgtaaggtgtgaggaaggtggggatgacgtcaaatcagcacggcccttacgtccggggctacacacgtgttacaatggcaggtacagagagacggtyscyygyaaagtsgatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcctagggcgaaactggtaattggggctaagtcgtaacaaggtaacc

SEQ ID NO: 19 - RNA, 1468 nt, Alistipes putredinis
  source 1..1468: mol_type = rRNA
cnatggagagtttgatcctggctcaggatnaacgctagcggcaggcttaacacatgcaagtcgaggggcagcatcatggatagcaatatctatggtcggcgaccggcgcacgggtgcgtaacgcgtatgcaacctacctttaacagggggataacaccgagaaattggtactaataccccataatatcatagaaggcatctnttatggttgaaaattccgatggttagagatgggcatgcgttgtattagctagttggtggggtaacggctcaccaaggcaacgatacatagggggactgagaggttaaccccccacactggtactgagacacggaccngactcctacgggaggcagcagtgaggaatattggtcaatggacgcaagtctgaaccagccatgccgcgtgcaggatgacggctctntgagttgtaaactncttttgtacgagggtaaacgcagatacgtgtatctgtctgaaagtatcgtacgaataaggatcggctaactccgtgccagcagccgcngtnatacggaggattcnagcgttatccggatttattgggtttaaagggtgcgtaggcggtttgataagttagaggtgaaatttcggggctcaaccctgaacgtgcctctnatactgttgagctagagagtagttgcggtaggcggaatgtatggtgtagcggtgaaatgcttagagatcatacagaacaccgattgcgaaggcagcttaccnaactatatctgacgctgatgcnnnaaagcgtngggagcaaacaggattagataccctggtagtccacgcagtaaacgatgataactcgttgtcggcgatacacagtcggtgactaagcgaaagcgatnagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttccttcggggcgcgaaactaggtgctgcatggttgtcgtcagctcgtgccgtgaggtgtcgggttaagtcccataacgagcgcaacccctaccgttagttgccatcagntgaagctgggcnctctggcgggactgccggtgtaagccgagaggaaggtggggatgacgtcaaatcagcacggcccntacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgcctnaagttcgtgaccgcaaggagcgacctagggcaaaactggtgac

SEQ ID NO: 20 - RNA, 1488 nt, Bifidobacterium bifidum
  source 1..1488: mol_type = rRNA
gatgaacgctggcggcgtgcttaacacatgcaagtcgaacgggatccatcaagcttgcttggtggtgagagtggcgaacgggtgagtaatgcgtgaccgacctgccccatgctccggaatagctcctggaaacgggtggtaatgccggatgttccacatgatcgcatgtgattgtgggaaagattctatcggcgtgggatggggtcgcgtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacaggattagataccctggtagtccacgccgtaaacggtggacgctggatgtggggcacgttccacgtgttccgtgtcggagctaacgcgttaagcgtcccgcctggggagtacggccgcaaggctaaaactcaaagaaattgacgggggcccgcacaagcggcggagcatgcggattaattcgatgcaacgcgaagaaccttacctgggcttgacatgttcccgacgacgccagagatggcgtttcccttcggggcgggttcacaggtggtgcatggtcgtcgtcagctcgtgtcgtgagatgttgggttaagtcccgcaacgagcgcaaccctcgccccgtgttgccagcacgttatggtgggaactcacgggggaccgccggggttaactcggaggaaggtggggatgacgtcagatcatcatgccccttacgtccagggcttcacgcatgctacaatggccggtacagcgggatgcgacatggcgacatggagcggatccctgaaaaccggtctcagttcggatcggagcctgcaacccggctccgtgaaggcggagtcgctagtaatcgcggatcagcaacgccgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctg

SEQ ID NO: 21 - RNA, 1787 nt, Candida albicans
  source 1..1787: mol_type = rRNA
tatctggttgatcctgccagtagtcatatgcttgtctcaaagattaagccatgcatgtctaagtataagcaatttatacagtgaaactgcgaatggctcattaaatcagttatcgtttatttgatagtaccttactacttggataaccgtggtaattctagagctaatacatgcttaaaatcccgactgtttggaagggatgtatttattagataaaaaatcaatgccttcgggctctttgatgattcataataacttttcgaatcgcatggccttgtgctggcgatggttcattcaaatttctgccctatcaactttcgatggtaggatagtggcctaccatggtttcaacgggtaacggggaataagggttcgattccggagagggagcctgagaaacggctaccacatccaaggaaggcagcaggcgcgcaaattacccaatcccgacacggggaggtagtgacaataaataacgatacagggcccttttgggtcttgtaattggaatgagtacaatgtaaataccttaacgaggaacaattggagggcaagtctggtgccagcagccgcggtaattccagctccaaaagcgtatattaaagttgttgcagttaaaaagctcgtagttgaaccttgggcttggctggccggtccatctttttgatgcgtactggacccagccgagcctttccttctgggtagccatttatggcgaaccaggacttttactttgaaaaaattagagtgttcaaagcaggcctttgctcgaatatattagcatggaataatagaataggacgttatggttctattttgttggtttctaggaccatcgtaatgattaatagggacggtcgggggtatcagtattcagttgtcagaggtgaaattcttggatttactgaagactaactactgcgaaagcatttaccaaggacgttttcattaatcaagaacgaaagttaggggatcgaagatgatcagataccgtcgtagtcttaaccataaactatgccgactagggatcggttgttgttcttttattgacgcaatcggcaccttacgagaaatcaaagtctttgggttctggggggagtatggtcgcaaggctgaaacttaaaggaattgacggaagggcaccaccaggagtggagcctgcggcttaatttgactcaacacggggaaactcaccaggtccagacacaataaggattgacagattgagagctctttcttgattttgtgggtggtggtgcatggccgttcttagttggtggagtgatttgtctgcttaattgcgataacgaacgagaccttaacctactaaatagtgctgctagcatttgctggtatagtcacttcttagagggactatcgacttcaagtcgatggaagtttgaggcaataacaggtctgtgatgcccttagacgttctgggccgcacgcgcgctacactgacggagccagcgagtataagccttggccgagaggtctgggaaatcttgtgaaactccgtcgtgctggggatagagcattgtaattgttgctcttcaacgaggaattcctagtaagcgcaagtcatcagcttgcgttgattacgtccctgccctttgtacacaccgcccgtcgctactaccgattgaatggcttagtgaggcctccggattggtttaggaaagggggcaacctcattctggaaccgagaagctggtcaaacttggtcatttagaggaagtaaaagtcgtaacaaggtttccgtaggtgaacctgcggaaggatcatta

SEQ ID NO: 22 - RNA, 593 nt, Candida albicans
  source 1..593: mol_type = rRNA
atatcaataagcggaggaaaagaaaccaacagggattgcctcagtagcggcgagtgaagcggcaaaagctcaaatttgaaatctggcgtctttggcgtccgagttgtaatttgaagaaggtatctttgggcccggctcttgtctatgttccttggaacaggacgtcacagagggtgagaatcccgtgcgatgagatgacccgggtctgtgtaaagttccttcgacgagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaagggcttgagatcagacttggtattttgcatgttgctctctcgggggcggccgctgcggtttaccgggccagcatcggtttggagcggcaggataatggcggaggaatgtggcacggcttctgctgtgtgttatagcctctgacgatactgccagcctagaccgaggactgcggtttttacctaggatgttggcataatgatcttaagtcgc

SEQ ID NO: 23 - RNA, 593 nt, Saccharomyces cerevisiae; identical to SEQ ID NO: 22
  source 1..593: mol_type = rRNA

SEQ ID NO: 24 - RNA, 3284 nt, Saccharomyces cerevisiae
  source 1..3284: mol_type = rRNA
aaaccaaccgggattgccttagtaacggcgagtgaagcggcaaaagctcaaatttgaaatctggtaccttcggtgcccgagttgtaatttggagagggcaactttggggccgttccttgtctatgttccttggaacaggacgtcatagagggtgagaatcccgtgtggcgaggagtgcggttctttgtaaagtgccttcgaagagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaagggcatttgatcagacatggtgttttgtgccctctgctccttgtgggtaggggaatctcgcatttcactgggccagcatcagttttggtggcaggataaatccataggaatgtagcttgcctcggtaagtattatagcctgtgggaatactgccagctgggactgaggactgcgacgtaagtcaaggatgctggcataatggttatatgccgcccgtcttgaaacacggaccaaggagtctaacgtctatgcgagtgtttgggtgtaaaacccatacgtgtaatgaaagtgaacgtaggttggggcctcgcaagaggtgcacaatcgaccgatcctgatgtcttcggatggatttgagtaagagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttcctgccgaagtttccctcaggatagcagaagctcgtatcagttttatgaggtaaagcgaatgattagaggttccggggtcgaaatgaccttgacctattctcaaactttaaatatgtaagaagtccttgttacttaattgaacgtggacatttgaatgaagagcttttagtgggccatttttggtaagcagaactggcgatgcgggatgaaccgaacgtagagttaaggtgccggaatacacgctcatcagacaccacaaaaggtgttagttcatctagacagccggacggtggccatggaagtcggaatccgctaaggagtgtgtaacaactcaccggccgaatgaactagccctgaaaatggatggcgctcaagcgtgttacctatactctaccgtcagggttgatatgatgccctgacgagtaggcaggcgtggaggtcagtgacgaagcctagaccgtaaggtcgggtcgaacggcctctagtgcagatcttggtggtagtagcaaatattcaaatgagaactttgaagactgaagtggggaaaggttccacgtcaacagcagttggacgtgggttagtcgatcctaagagatggggaagctccgtttcaaaggcctgattttatgcaggccaccatcgaaagggaatccggttaagattccggaacctggatatggattcttcacggtaacgtaactgaatgtggagacgtcggcgcgagccctgggaggagttatcttttcttcttaacagcttatcaccccggaattggtttatccggagatggggtcttatggctggaagaggccagcacctttgctggctccggtgcgcttgtgacggcccgtgaaaatccacaggaaggaatagttttcatgccaggtcgtactgataaccgcagcaggtctccaaggtgaacagcctctagttgatagaataatgtagataagggaagtcggcaaaatagatccgtaacttcgggataaggattggctctaagggtcgggtagtgagggccttggtcagacgcagcgggcgtgcttgtggactgcttggtggggcttgctctgctaggcggactacttgcgtgccttgttgtagacggtcttggtaggtctcttgtagaccgtcgcttgctacaattaacgatcaacttagaactggtacggacaaggggaatctgactgtctaattaaaacatagcattgcgatggtcagaaagtgatgttgacgcaatgtgatttctgcccagtgctctgaatgtcaaagtgaagaaattcaaccaagcgcgggtaaacggcgggagtaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaagagacatagagggtgtagaataagtgggagcttcggcgccagtgaaataccactacctttatagtttctttacttattcaatgaagcggagctggaattcattttccacgttctagcattcaaggtcccattcggggctgatccgggttgaagacattgtcaggtggggagtttggctggggcggcacatctgttaaacgataacgcagatgtcctaaggggggctcatggagaacagaaatctccagtagaacaaaagggtaaaagcccccttgattttgattttcagtgtgaatacaaaccatgaaagtgtggcctatcgatcctttagtccctcggaatttgaggctagaggtgccagaaaagttaccacagggataactggcttgtggcagtcaagcgttcatagcgacattgctttttgattcttcgatgtcggctcttcctatcataccgaagcagaattcggtaagcgttggattgttcacccactaatagggaacgtgagctgggtttagaccgtcgtgagacaggttagttttaccctactgatgaatgttaccgcaatagtaattgaacttagtacgagaggaacagttcattcggataattggtttttgcggctgtctgatcaggcattgccgcgaagctaccatccgctggattatggctgaacgcctctaagtcagaatccatgctagaacgcggtgatttctttgctccacacaatatagatggatacgaataaggcgtccttgtggcgtcgctgaaccatagcaggctagcaacggtgcacttggcggaaaggccttgggtgcttgctggcgaattgcaatgtcattttgcgtggggataaatcatttgtatacgacttagatgtacaacggggtattgtaagcagtagagtag